(19) United States
(12) Patent Application Publication
(10) Pub. No.: US 2017/0189313 A1
(43) Pub. Date: Jul. 6, 2017

(54) COMPOSITIONS CONTAINING POLYCARBODIIMIDES AND LATEX POLYMERS FOR TREATING KERATINOUS SUBSTRATES

(71) Applicant: L'OREAL, Paris (FR)

(72) Inventors: Siliu TAN, Westfield, NJ (US); Charles Michael Sanford SHAW, Madison, NJ (US); Nghi VAN NGUYEN, Edison, NJ (US); Jim Mitchell SINGER, South Orange, NJ (US); XianZhi ZHOU, Millburn, NJ (US); Aditi GOGINENI, Rahway, NJ (US)

(21) Appl. No.: 15/396,083

(22) Filed: Dec. 30, 2016

Related U.S. Application Data
(63) Continuation-in-part of application No. 14/986,019, filed on Dec. 31, 2015.
Application No. 14/986,019 is a continuation-in-part of application No. PCT/US2016/069536, filed on Dec. 30, 2016.

(51) Int. Cl. A61K 8/88; A61Q 5/00; A61K 8/81

(52) U.S. Cl. CPC A61K 8/88; A61K 8/8141; A61Q 5/002; A61K 2800/594; A61K 2800/882; A61K 2800/884

(57) ABSTRACT

Compositions including polycarbodiimide and derivatives thereof together with latex polymers and derivatives thereof to enhance the quality of the keratinous substrates. The present invention relates to a cosmetic treatment and process for treating keratinous materials, in particular for hair-care and hair-styling. A method of styling or shaping hair is also disclosed.

### CROSS-REFERENCE TO RELATED APPLICATION

This patent application claims the benefit of U.S. Non-Provisional patent application Ser. No. 14/986,019 filed on Dec. 31, 2015, and entitled “COMPOSITIONS CONTAINING POLYCARBODIIMIDES AND LATEX POLYMERS FOR TREATING KERATINOUS SUBSTRATES,” the disclosure of which is incorporated by reference as if fully rewritten herein.

### FIELD OF THE INVENTION

The present invention generally relates to a composition and method for treating keratinou substrates such as hair, skin and nails. More particularly, the present invention relates to keratinous treatment compositions having a polycarbodiimide compound, and, in certain embodiments, a latex polymer, for durable non-permanent shaping or for durable retention of a non-permanent shape of at least one keratinous fiber and providing protection from extrinsic damage or repair of keratinous fibers such as hair.

### BACKGROUND OF THE INVENTION

The appearance and/or condition of keratinous substrates, for example, keratinous fibers such as hair, skin, nails, and lips, are often affected by both extrinsic and intrinsic factors such as aging. In particular, when keratinous substrates are exposed to environmental conditions, for example, high or low humidity or to ultraviolet radiation from the sun, these substrates can lose many of their desirable properties and even become damaged. Keratinous fibers, especially hair, are constantly exposed to harsh extrinsic conditions, such as sun, chemical damage, e.g., from detergents, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. Furthermore, hair can lose its desirable attributes due to physical stress on the hair such as brushing and application of heat. The magnitude of the consequences of these factors is variable, depending on, for example, the quality of the hair, length, style, and environmental factors. As such, these factors generally result in damage to the keratinous fibers, either by affecting protective materials on the surface of the hair (the cuticle), or by altering the hair fiber internally (the cortex). More specifically, extrinsic conditions may strip protective materials from the surface of the hair, and/or they may disrupt the organized structure of the hair fibers, called the α-structure, which may be accompanied by a decrease in the tensile strength. Such damage to hair by extrinsic factors is more evident the further the hair fiber has grown from the root, because the hair has been exposed longer to such extrinsic factors. In effect, the hair has what may be called a “damage history” as it grows, i.e., the further from the root, the lower the tensile strength and the greater the breakdown in α-structure that has occurred. As a result, consumers continue to seek products such as hair care and hair cosmetic compositions which protect and enhance the appearance of hair as well as reduce the deleterious effects of adverse environmental conditions, photo-damage, and physical stress.

Morphologically, a hair fiber contains four structural units: cuticle, cortex, medulla, and intercellular cement. Robbins, C. R. Chemical and Physical Behavior of Human Hair, 3rd Edition, Springer-Verlag (1994). The cuticle layers are located on the hair surface and consist of flat overlapping cells (“scales”). These scales are attached at the root end and point toward the distal (tip) end of the fiber and form layers around the hair cortex. The cortex comprises the major part of the hair fiber. The cortex consists of spindle-shaped cells, or macrofibrils, that are aligned along the fiber axis. The macrofibrils further consist of microfibrils (highly organized protein units) that are embedded in the matrix of amorphous protein structure. The medulla is a porous region in the center of the fiber. The medulla is a common part of wool fibers but is found only in thicker human hair fibers. Finally, the intercellular cement is the material that binds the cells together, forming the major pathway for diffusion into the fibers.

The mechanical properties of hair are determined by the cortex. A two-phase model for the cortex organization has been suggested. Milczarek et al, Colloid Polym. Sci., 270, 1106-1115 (1992). In this model, water-impenetrable microfilaments (“rods”) are oriented parallel with the fiber axis. The microfilaments are embedded in a water-penetrable matrix (“cement”). Within the microfilaments, coiled protein molecules are arranged in a specific and highly organized way, representing a degree of crystallinity in the hair fiber.

Similar to other crystalline structures, hair fibers display a distinct diffraction pattern when examined by wide-angle X-ray diffraction. In normal, non-stretched hair fibers this pattern is called an “alpha-pattern”. The alpha-pattern or α-structure of hair is characterized by specific repeated spacings (9.8 Å, 5.1 Å, and 1.5 Å). All proteins that display this X-ray diffraction pattern are called α-proteins and include, among others, human hair and nails, wool, and porcupine quill. When the hair fiber is stretched in water, a new X-ray diffraction pattern emerges that is called a “β-pattern”, with new spacings (9.8 Å, 4.65 Å, and 3.3 Å).

Damage to hair may occur in the cuticle and/or the cortex. When normal hair is damaged by heat, chemical treatment, UV radiation, and/or physical/mechanical means, myriad chemical and physical changes are induced in the hair. For example, these damaging processes have been known to produce removal or damage to cuticle scales or to cleave the thioester linkage holding the hydrophobic 18-methyl eicosanoic acid (“18-MEA”) layer to hair. Thus, it is commonly observed that undamaged hair exhibits significant hydrophobic character, whereas damaged hair shows significant hydrophilic character due to the removal of surface lipids.

There is a need, therefore, for cosmetic products that are useful in protecting the chemical and physical structure of keratinous fibers from harsh extrinsic conditions and restoring the hair's physical properties to undamaged states following damage by extrinsic conditions. More particularly, there is a need to find materials or compositions or methods that can provide a water-resistant and/or hydrophobic and/or protective barrier to hair to protect it at the cortex. Such a protective barrier should not be easily transferred from the substrate over time by normal everyday activity. In addition, the protective barrier should be shampoo, wash or water-resistant so that the barrier is not easily removed. Non-transfer and shampoo, wash or water-resistant cosmetic, hair and skin care compositions are sought which have the advantage of forming a deposit which does not undergo even partial transfer to the substrates with which they are brought into contact (for example, clothing). It is also desirable to have compositions that do not easily “run off” or wash off the skin and lips when exposed to water, rain or tears. Accordingly, a product that provides a protective barrier to the substrate that also is shampoo, wash or water resistant and non-transferable would be of benefit to the area of cosmetic products. As such, makers of cosmetic products such as hair and skin care products continue to seek materials and ingredients that can provide such benefits.

In addition, in today's market, many consumers prefer the flexibility of non-permanent hairstyles, that is, those styles obtained via non-permanent shaping of the hair. Typically, such non-permanent styles disappear when the hair is wetted, especially when the hair is washed with water and/or shampoo or when the hair is exposed to high humidity conditions. Methods for non-permanent shaping of keratinous fibers include, for example, brushing, teasing, braiding, the use of hair rollers, and heat styling, optionally with a commercially available styling product. Non-limiting examples of heat styling include blow drying, crimping, curling, and straightening methods using elevated temperatures (such as, for example, setting hair in curlers and heating, and curling with a curling iron and/or hot/steam rollers and/or flat iron).

While such compositions and methods may provide for non-permanent shaping of keratinous fibers, many consumers also desire longer lasting or durable styling/shaping than most known materials (e.g., film-forming agents, resins, gums, and/or adhesive polymers), commercially available products (e.g., conventional hair sprays, mousses, gels and lotions), and methods employing these materials and products provide. For example, many consumers desire compositions and methods that improve and preserve non-permanent curl formation or hair style.

Further, many people desire compositions and methods for retaining a particular non-permanent shape or style of keratinous fibers such as hair. A common way to retain a particular hairstyle is with the use of a hairspray, typically applied after styling the hair. Other methods to retain a hairstyle or shape of keratinous fibers include the use of mousses, gels, and lotions. The materials in these compositions are generally film forming agents, resins, gums, and/or adhesive polymers.

There is a need, therefore, for materials, compositions and methods that result in more durable or longer lasting shape or style even when the styled/shaped/curled hair is exposed to adverse environmental and physical factors and/or when wetted, washed, or shampooed.

To achieve at least one of these and other advantages, the present invention provides a composition that includes a polycarbodiimide compound and a latex polymer and a method of styling or shaping a keratinous substrate, and more particularly, a keratinous fiber chosen from hair, eyelashes and eyebrows, by applying to the keratinous fiber said composition.

The present invention also provides for protecting and/or repairing a keratinous substrate, and more particularly, a keratinous fiber chosen from hair, eyelashes and eyebrows from extrinsic damage caused by heating, UV radiation or chemical treatment by applying to said keratinous fiber a composition that includes a polycarbodiimide compound and a latex polymer in an amount effective to confer or improve the keratinous fiber's hydrophobicity.

### BRIEF SUMMARY OF THE INVENTION

In an exemplary embodiment, a keratinous treatment composition including a polycarbodiimide compound and a latex polymer is disclosed. The composition includes about 0.01 to about 20.0%, by weight, based on the total weight of the composition, of a combined amount of the polycarbodiimide compound and the latex polymer. The composition includes amounts of each of the polycarbodiimide compound and the latex polymer sufficient to impart one or more of increased hydrophobicity to the keratinous substrate after application thereto and increased hold to the shape or configuration of the keratinous substrate. In some particular embodiment, the composition includes at least two latex polymers together with the polycarbodiimide compound.

In another exemplary embodiment, a method of styling or shaping a keratinous fiber chosen from hair, eyelashes and eyebrows is disclosed, wherein the method includes applying to the keratinous fiber a composition including the polycarbodiimide and the latex polymer. In some embodiments, the polycarbodiimide and the latex polymer are each in amounts sufficient to impart a hold to the shape or configuration of the keratinous fiber.

In yet another exemplary embodiment, a method of protecting a keratinous substrate chosen from hair, eyelashes and eyebrows from extrinsic damage caused by heating, UV radiation or chemical treatment, or of repairing a keratinous substrate chosen from hair, eyelashes and eyebrows following extrinsic damage caused by heating, UV radiation or chemical treatment is disclosed. The method includes applying to said keratinous fiber a composition including the polycarbodiimide and the latex polymer in an amount effective to protect or repair said keratinous fiber; wherein the polycarbodiimide compound is present at a concentration of from about 00.1 to about 10% by weight, based on the total weight of the composition.

Another embodiment of the present invention is a method of protecting a keratinous substrate chosen from hair comprising applying to the keratinous fiber the composition of the present invention in an amount effective to protect or repair said keratinous substrate before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

In some embodiments, the keratinous substrate in the above described composition is heated and the composition is applied to the substrate prior to heating or during heating or after heating the substrate.

In another exemplary embodiment, a method for durable non-permanent shaping of at least one keratinous fiber or for durable retention of a non-permanent shape of at least one keratinous fiber is disclosed. The method includes applying to said at least one keratinous fiber the composition including the polycarbodiimide and the latex polymer.

In some embodiments, the above-described method includes a step of heating the keratinous fiber prior to or during or after the application of the above-described composition.

The latex polymer in the compositions of the present disclosure are chosen from carboxyl functional latex polymers. These carboxyl functional latex polymers may be selected from acrylate latex polymers, polyurethane latex polymers, silicone latex polymers, and mixtures thereof.

The present invention is also directed to a method for cosmetic treatment of keratinous tissues, such as keratinous fibers, by applying the above-disclosed composition onto a surface of the keratinous tissue, such as the cuticle of hair fibers.

The present invention is also directed to methods for applying the above-disclosed composition onto a surface of the keratinous tissue, such as the cuticle of hair fibers in a step wise fashion, the steps including a one step process and a multi-step process including two, three or more steps, and combinations of these.

According to some such embodiments, the method includes a one-step process, wherein a composition including the polycarbodiimide, latex polymer, and solvent is prepared and applied onto the keratinous substrate.

According to other embodiments, the method includes a two-step application process, wherein individual components (polycarbodiimide combined with a solvent and latex polymer combined with a solvent) are applied to the keratinous substrate in a step-wise fashion in any order to treat the substrate. In one such embodiment, the keratinous substrate is first treated with the polycarbodiimide phase, followed by treatment with the latex polymer phase.

According to other embodiments, the method includes a reverse-step system, wherein compositions comprising at least one polycarbodiimide and at least one latex polymer are prepared as separate phases, and each phase is applied separately to the keratinous substrate, such as hair. In one such embodiment, the keratinous substrate is first treated with the latex polymer phase, followed by treatment with the polycarbodiimide phase.

The present invention is also directed to methods and kits for cosmetic treatment of keratinous tissues, such as keratinous fibers, by applying the above-disclosed composition onto a surface of the keratinous tissue, such as the cuticle of hair fibers in a step wise fashion. According to some such embodiments, the kit includes separate packaging of one or more of the actives of the inventive composition provided in one or more of thickened or un-thickened aqueous and non-aqueous phases, and packaging of any of the foregoing with one or more of processing agents selected from a coloring agent, a pigmenting agent, a permanent process agent, a relaxing process agent, a straightening process agent, and a highlighting process agent.

Other features and advantages of the present invention will be apparent from the following more detailed description of the preferred embodiment which illustrates, by way of example, the principles of the invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

The term “comprising” (and its grammatical variations) as used herein is used in the inclusive sense of “having” or “including” and not in the exclusive sense of “consisting only of.”

“Conditioning,” as used herein, means imparting at least one of combability, manageability, moisture-retentivity, luster, shine, softness, and body to the hair.

“Durable conditioning,” as used herein, means that, following at least one shampoo/washing/rinsing after treatment of keratinous fibers such as hair with the compositions of the present disclosure, treated hair still remains in a more conditioned state as compared to untreated hair. The state of conditioning can be evaluated by measuring, and comparing, the ease of combability of the treated hair and of the untreated hair in terms of combing work (gm-in) and/or the substantivity of the conditioning agent on the hair and/or the hydrophobicity of hair which can be assessed by contact angle measurements (spread of a water droplet on the surface of the hair).

“Durable retention of a shape,” as used herein, means that, following at least one shampoo/washing/rinsing or exposure to high humidity conditions after treatment of keratinous fibers such as hair with the compositions of the present disclosure, treated hair still exhibits the ability to retain a particular or desirable shape after styling as compared to the exhibited ability of untreated hair to retain a particular or desirable shape after styling. “Durable retention of a shape” can also be related to the hydrophobicity of hair which can be assessed by contact angle measurements (spread of a water droplet on the surface of the hair).

“Durable shaping,” as used herein, refers to holding or keeping a shape of a keratinous fiber until the keratinous fiber is washed with water and/or shampoo or exposed to high humidity conditions. Retention of a shape can be evaluated by measuring, and comparing, the ability to retain a curl under conditions of high relative humidity of the treated hair and of the untreated hair in terms of Curl Efficiency. “Durable shaping” can also be related to imparting hydrophobicity to hair which can be assessed by contact angle measurements (spread of a water droplet on the surface of the hair).

“Heating” refers to the use of elevated temperature (i.e., above room temperature such as above 40° C.). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hood dryers, heating caps and steamers.

“A heat-activated” composition, as used herein, refers to a composition which, for example, shapes the at least one keratinous fiber better than the same composition which is not heated during or after application of the composition. Another example includes a composition which retains a shape of at least one keratinous fiber better than the same composition which is not heated during or after application.

“High humidity,” as defined herein, refers to atmospheric humidity above 40%.

The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “style” or styling” as used herein includes shaping, straightening, curling, or placing a keratin fiber such as hair, in a particular arrangement, form or configuration; or altering the curvature of a keratinous fiber or other substrate; or re-positioning a keratin fiber or other substrate to a different arrangement, form or configuration; or providing/maintaining a hold to the shape or configuration of the keratin fiber. In some embodiments, the hold to the shape of configuration of the fiber may be expressed as an improved bending force property.

As used herein, the terms “styling keratinous fibers” and variations thereof are understood to refer to any means or method of modifying the appearance of the keratinous fibers or the hair with respect to their spatial arrangement or configuration or curvature or form. When the keratinous fibers comprise hair on the human head, the term “styling keratinous fibers” or “styling hair” is also understood to include curling or waving or embossing the hair or smoothing or straightening the hair, or spiking the hair or providing/maintaining a hold to the shape or configuration of the keratin fiber.

The term “treat” (and its grammatical variations) as used herein refers to the application of the compositions of the present invention onto keratinous substrates such as keratinous fibers or hair or skin.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It is an object of the present invention to provide materials and compositions and methods which provide both a protective barrier or coating onto keratinous substrates such as hair which impart beneficial physical properties-such as hydrophobicity, ease of combing, etc.—to hair, in particular, damaged hair, as well as impart durable or long lasting physical properties which can even mimic properties of natural/undamaged hair.

It is also an object of the present invention to provide materials and compositions and methods which improve the resistance of the keratinous substrate to humidity and other environmental conditions as well as to cleansing/washing so as to preserve a non-permanent shape or style or for durable retention of a non-permanent shape or style of the keratinous substrate such as hair. Furthermore, it is an object of the present invention to provide materials and compositions and methods which provide a protective barrier or coating onto keratinous substrates, e.g. hair, that imparts a hold to the shape or style or configuration of the hair.

It has been surprisingly and unexpectedly discovered by the inventors that a composition containing the combination of a latex polymer and polycarbodiimides for cosmetic application, when applied to keratinous substrates such as hair, can impart styling or shaping benefits to the substrates that can be characterized by a range of hold (i.e., from light to medium to strong styling hold).

It has also been surprisingly and unexpectedly discovered by the inventors that the above-described composition, when applied to keratinous substrates such as hair, enhance the properties of the hair such as with respect to hydrophobicity, adhesion, chemical resistance, water resistance etc. and deliver superior performance to the substrate.

In embodiments of this disclosure, the combination of polycarbodiimide compounds with a latex polymer enhance the properties of hair wherein the combination increases the conditioning effect (e.g., hydrophobicity, shine and smoothness), strengthens the hair, increases the stiffness and humidity resistance of hair and ameliorates the condition of damaged hair by improving the appearance and quality of hair (for example, smoother feel, softer feel, less frizzy, less dry, more discipline). In addition, the composition, according to the present disclosure, provides a significant reduction in combing force which indicates that hair is easier to comb and/or has less tangling.

The compositions according to the present disclosure imparted increased percent curl retention on curled/shaped hair indicating higher resistance of the hair to high humidity and high temperature as compared to compositions with either material alone.

It was also surprisingly and unexpectedly discovered by the inventors that curled/shaped hair treated with the compositions according to the present disclosure maintained its shape (i.e., same or similar degree of curl retention) even after shampooing the hair.

It has also been surprisingly and unexpectedly discovered by the inventors that sequential application of each of the actives selected from one or more polycarbodiimides and latex polymers, in some embodiments, provides enhanced results as compared to application of some combinations of the two actives together when applied to keratinous substrates comprising hair. The benefits thus conferred by such sequential application include one or more of enhanced ease of brushing and lasting fizz in humidity control.

It has also been surprisingly and unexpectedly discovered by the inventors that hair treated with inventive compositions containing two or more latex polymers, for example a combination of cationic and non-ionic latex polymers, together with and Polycarbodiimide, exhibited increased ease of brushing, initial frizz control after brushing and lasting frizz control effects when exposed to high humidity conditions. Further, as compared to compositions having only one latex polymer and polycarbodiimide, compositions having two latex polymers and polycarbodiimide surprisingly and unexpectedly exhibit greater shampoo resistance.

It was also found that the interactions between the polycarbodiimide and the latex polymers at certain weight ratios resulted in compositions that formed a film or coating having different physical and mechanical properties such that when applied on keratinous substrate, for example hair, enabled one to achieve different styling/shaping holds on the substrate.

Without being bound to any one theory, the inventors of the present disclosure believe that the polycarbodiimide compound and the latex polymer chosen from carboxyl functional acrylate latex polymers, carboxyl functional polyurethane latex polymers, carboxyl functional silicone latex polymers and mixtures thereof comprising the keratinous treatment compositions of the invention react to each other and to the keratin substrate when such compositions are applied onto keratinous substrates such as hair, nails or skin. It is also believed that the compositions of the present disclosure provide a protective barrier useful in cosmetic applications such as hair care, hair styling, nail care, makeup, skin care, and sun care products such that the hydrophobicity of the keratinous substrates is improved or restored resulting in significantly better cosmeticities, feel and appearance, and less damaged condition of the substrates such as hair and skin.

Other subjects, characteristics, aspects and advantages of embodiments of the disclosure will emerge even more clearly on reading the description and the various examples that follow.

The range of concentrations by weight of the composition over which the association provides beneficial and cosmetic properties to keratinous substrates (for example, hydrophobicity or hold) is about 0.01% to about 20%, or about 0.05% to about 10%, or about 0.1% to about 5%, by weight, including all ranges and subranges there-between, based on the total weight of the composition, of a combined amount of the polycarbodiimide compound and latex polymer.

In accordance with some embodiments, the range of the weight ratio of the polycarbodiimide to the latex polymer in the composition over which their association provides hydrophobicity to and/or hold to the shape or configuration of keratinous substrates such as hair, is from about 1:50 about 20:1 or about 1:40 to about 10:1 or about 20:1 to about 10:1 or about 1:10 to about 10:1 or about 1:2 to about 2:1 or about 1:5 to about 5:1, including all ranges and subranges there-between, or such as about 1:1 or about 1:2 or about 1:3 or about 1:4 or about 1:5 or about 1:7 or about 1:7 or about 1:8 or about 1:9 or about 1:10 or about 1:20 or about 1:40 or about 1:50 or about 50:1 or about 40:1 or about 20:1 or about 10:1 or about 9:1 or about 8:1 or about 7:1 or about 8:1 or about 7:1 or about 6:1 or about 5:1 or about 4:1 or about 3:1 or about 2:1.

In some embodiments, the composition comprising the polycarbodiimide and the latex polymer can form a film when applied to a substrate. The film may, according to at least certain embodiments of the disclosure, have a Young's modulus ranging from about 0.05 MPa to about 5 GPa, and/or a strain, under stress at 0.6 MPa, that ranges up to about greater than 50%, such as up to about 300%. By way of example only, the film may have a Young's modulus ranging from about 500 MPa to about 5 GPa and a strain, under stress at 0.6 MPa, ranging from about 0.001% to less than about 1%.

By way of further example, the film may have a Young's modulus ranging from about 100 MPa to about 800 MPa, or from about 100 MPa to about 700 MPa, or from about 150 MPa to about 675 MPa. In some examples, the film may have a Young's modulus of about 150 MPa, or of about 175 MPa, or of about 200 MPa, or of about 250 MPa, or of about 300 MPa, or of about 350 MPa, or of about 400 MPa, or of about 450 MPa, or of about 500 MPa, or of about 550 MPa, 600 MPa, or of about 650 MPa, or of about 700 MPa, or of about 750 MPa, or of about 800 MPa.

In some examples, the film formed according to compositions having a ratio of polycarbodiimide to latex polymer of from about 1:1 to about 1:3 to about 1:5 have a Young's modulus in the range from about 175 MPa to about 675 MPa, wherein in some such embodiments, the Young's modulus is greater as the ratio of polycarbodiimide to latex polymer approaches 1:1.

By way of further example, the film may have a Young's modulus ranging from about 0.05 MPa to about 100 MPa and a strain, under stress at 0.6 MPa, ranging from about 0.1% to greater than about 50% or from about 0.1% to about 1%. By way of yet further example, the film may have a Young's modulus ranging from about 0.05 MPa to about 10 MPa and a strain, under stress at 0.6 MPa, ranging from about 1% to greater than about 50% or from about 1% to about 5%.

Polycarbodiimides

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

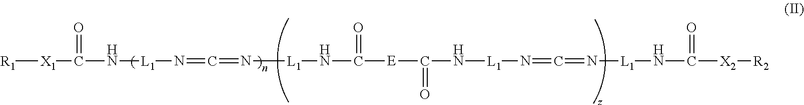

wherein X₁ and X₂ each independently represent O, S or NH. R₁ and R₂ are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L₁ (Linker of carbodiimide groups) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group, and a C₃ to C₁₂ divalent heterocyclic group; wherein a plurality of L₁s may be identical to or different from one another, and wherein in another embodiment, L₁ of formula (II) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group that is not chosen from m-tetramethylxylylene, and a C₃ to C₁₂ divalent heterocyclic group; wherein a plurality of L₁s may be identical to or different from one another;

wherein E is a radical selected from the following formulas:

O—R₃—O; S—R₄—S; and

R₅—N—R₄—N—R₅;

wherein R₃ and R₄ are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R₅ is hydrogen, or a hydrocarbon radical which can contain halogen atoms or one or more catenary (i.e.; in chain, bonded only to carbon) or non-catenary hetero atoms.

Examples of R₁ and R₂ can be methyl glycolate, methyl lactate, polypropylene glycol, polyethylene glycol monomethyl ether, dialkylamino alcohol.

Examples of L₁ can be the diradical of tolylene, hexamethylene, hydrogenated xylylene, xylylene, 2,2,4-trimethylhexamethylene, 1,12-dodecane, norbornane, 2,4-bis-(8-octyl)-1, 3-dioctylcyclobutane, 4,4′-dicyclohexylmethane, tetramethylxylylene, isophorone, 1,5-naphthylene, 4, 4′ diphenylmethane, 4, 4′ diphenyldimethylmethane, phenylene.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

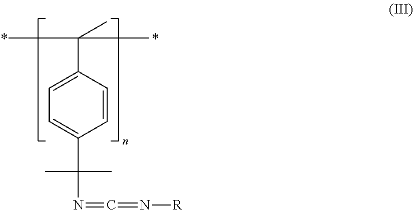

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

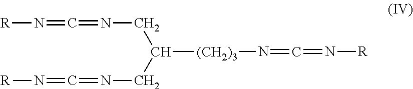

wherein R is an alkyl, cycloalkyl or aryl group (in some particular embodiments having from 1 to 24 carbon atoms).

In one embodiment, the compositions of the present disclosure does not employ a polycarbodiimide having a linker L₁ chosen from m-tetramethylxylylene.

Suitable polycarbodiimide compounds include, but are not limited to, those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide of the present disclosure is selected from compounds of formula (II) wherein L₁ (Linker of carbodiimide groups) represents a C₁ to C₁₈ divalent aliphatic hydrocarbon group, a C₃ to C₁₃ divalent alicyclic hydrocarbon group, a C₃ to C₁₂ divalent heterocyclic group, or a C₆ to C₁₄ divalent aromatic hydrocarbon group;

wherein a plurality of L₁s may be identical to or different from one another.

In other embodiments, the polycarbodiimide of the present disclosure is selected from compounds of formula (II) wherein L₁ is not chosen from m-tetramethylxylylene.

In certain embodiments, the composition of the present disclosure is devoid of a polycarbodiimide that has a linker L₁ chosen from m-tetramethylxylylene.

In accordance with the various embodiments, the polycarbodiimide compound is present in the compositions according to the disclosure in amounts range from about 0.01% to about 10%, and in some embodiments from about 0.01% to about 8%, and in some further embodiments from about 0.01% to about 6%. In some representative embodiments, the polycarbodiimide compound is present from about 0.1% to about 5%, and from about 0.2% to about 3%. In yet other embodiments, the polycarbodiimide compound is present from at least 0.25%. And in yet other embodiments, the polycarbodiimide compound is present from at least 0.22% to up to about 10%. In some specific embodiments, the polycarbodiimide compound is present at about 3%, or about 2.75%, or about 2.5% or about 2%. In some specific embodiments, the polycarbodiimide compound is present at about 0.75%, or about 0.5%. In some other specific embodiments, the polycarbodiimide compound is present at about 0.25%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, to about 10 percent by weight, including increments and ranges therein and there between.

Latex Polymers

The composition according to the present disclosure comprises one or more latex polymers. These latex polymers may be chosen from carboxyl functional latex polymers which can be selected from acrylate latex polymers, polyurethane latex polymers, silicone latex polymers and mixtures thereof. The latex polymers chosen from carboxyl functional polymers can also be of the non-acrylate or non-polyurethane or non-silicone type of latex polymers.

In various embodiments, the one or more latex polymers of the present invention can be film-forming latex polymers or non film-forming latex polymers.

In various embodiments according to the disclosure, the latex polymers are present, as polymeric active material (dry weight basis), in an amount ranging from about 0.01% to about 10% by weight, or from about 0.01% to about 8% by weight, or from about 0.01% to about 6% by weight, including all ranges and subranges there between, based on the total weight of the composition. In some representative embodiments, the latex polymer is present from about 0.25% to about 10%, and from about 0.5% to about 5%. In yet other embodiments, the latex polymer is present from at least 0.5%. And in yet other embodiments, the latex polymer is present from at least 0.5% to up to about 40%. In some specific embodiments, the latex polymer is present at about 1.5%. In some other specific embodiments, the latex polymer is present at about 0.75%. In some other specific embodiments, the latex polymer is present at about 0.25%.

In other various embodiments, the latex polymers can be employed, as polymeric active material (dry weight basis), in an amount of about 0.01, 0.05 0.10, 0.15, 0.18, 0.2, 0.25, 0.30, 0.33, 0.37, 0.40, 0.45, 0.5, 0.55, 0.6, 0.67, 0.70, 0.75, 0.8, 0.85, 0.90, 0.95, 1, 1.2, 1.3, 1.4, 1.5, 1.6, 1.67, 1.70, 1.8, 1.9, 2, 2.2, 2.3, 2.4, 2.5, 2.6, 2.7, 2.8, 2.9, 3, 3.1, 3.2, 3.3, 3.4, 3.5, 3.6, 3.7, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.25, 6.5, 6.75, 7, 7.25, 7.5, 7.75, 8, 8.25, 8.5, 8.75, 9, 9.5 and 10 percent by weight, based on the total weight of the composition.

In at least certain embodiments of the disclosure, the one or more latex polymers are provided in the form of aqueous dispersions prior to formulating the compositions of the disclosure. In various embodiments, the aqueous dispersions may be obtained through an emulsion polymerization of monomers wherein the resulting latex polymers have a particle size lower than about 1 micron. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the latex polymers are produced from condensation reactions between monomers and subsequently dispersed in an aqueous medium.

Thus, the latex polymers may, in various exemplary embodiments, exist as dispersed polymer particles in a dispersion medium, such as an aqueous dispersion medium. The latex polymers may, in certain embodiments, each be dispersed in independent dispersion media. In yet further embodiments, the latex polymers may be dispersed together in the same dispersion medium.

The dispersion medium comprises at least one solvent chosen from water. The dispersion medium may further comprise at least one solvent chosen from cosmetically acceptable organic solvents. Cosmetically acceptable organic solvents may, in various embodiments, be water-miscible, e.g. capable of forming at 25° C. a homogeneous mixture that is transparent, or substantially transparent, to the eye. For instance, cosmetically acceptable organic solvents may be chosen from lower monoalcohols, such as those containing from about 1 to 5 carbon atoms, for example ethanol and isopropanol; polyols, including glycols, such as those containing from about 2 to 8 carbon atoms, for example propylene glycol, ethylene glycol, 1,3-butylene glycol, dipropylene glycol, hexylene glycol, and glycerin; hydrocarbons, such as, for example, isododecane and mineral oil; and silicones, such as dimethicones, cyclomethicones, and cyclopentasiloxane; as well as mixtures thereof.

In at least one embodiment, the solvent of the dispersion medium consists of water. In other embodiments, the solvent of the dispersion medium consists of water and at least one cosmetically acceptable organic solvent. In further embodiments, the solvent comprises water. In yet further embodiments, the solvent of the dispersion medium primarily comprises water. For example, the solvent of the dispersion medium may, in at least certain exemplary embodiments, comprise greater than 50% water, such as greater than 55% water, greater than 60% water, greater than 65% water, greater than 70% water, greater than 75% water, greater than 80% water, greater than 85% water, greater than 90% water, greater than 95% water, greater than 96% water, greater than 97% water, greater than 98% water, or greater than 99% water.

In embodiments according to the disclosure, the latex polymer (a) particles are not soluble in the solvent of the dispersion medium, i.e. are not water soluble and/or are not soluble in the at least one cosmetically acceptable organic solvent. Accordingly, the latex polymers retain their particulate form in the solvent or solvents chosen.

In at least certain exemplary embodiments, latex polymer (a) particles according to the disclosure may have an average diameter ranging up to about 1000 nm, such as from about 50 nm to about 800 nm, or from about 100 nm to about 500 nm. Such particle sizes may be measured with a laser granulometer (e.g. Brookhaven B190).

In various embodiments, the latex polymers may, independently, be neutralized, partially neutralized, or unneutralized. In exemplary embodiments where the latex polymers are neutralized or partially neutralized, the particle size may be, for example, greater than about 800 nm. In at least certain embodiments, the particulate form of the latex polymers is retained in the dispersion medium.

In further embodiments, the latex polymers may be chosen from uncharged and charged latex polymers. Thus, the latex polymers may, according to various exemplary embodiments, be chosen from nonionic latex polymers, cationic latex polymers, anionic latex polymers and amphoteric latex polymer.

As non-limiting examples of carboxyl functional latex polymers that may be used, mention may be made, independently, of acrylate latex polymers, polyurethane latex polymers and silicone latex polymers.

In certain embodiments, the latex polymer of the present invention is a carboxyl functional latex polymer chosen from acrylate latex polymers or polyurethane latex polymers, or mixtures thereof.

By way of non-limiting example only, the latex polymers may be chosen from acrylate latex polymers, such as those resulting from the homopolymerization or copolymerization of ethylenically unsaturated monomers chosen from monomers, (meth)acrylic monomers, (meth)acrylamide monomers and mono- and dicarboxylic unsaturated acids. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. Additional non-limiting examples of (meth)acrylic monomers include C1-C8 alkyl (meth)acrylic, such as, for example, methyl (meth)acrylic, ethyl (meth)acrylic, propyl (meth)acrylic, isopropyl (meth)acrylic, butyl (meth)acrylic, tert-butyl (meth)acrylic, pentyl(meth) acrylic, isopentyl (meth)acrylic, neopentyl (meth)acrylic, hexyl (meth)acrylic, isohexyl (meth)acrylic, 2-ethylhexyl (meth)acrylic, cyclohexyl (meth)acrylic, isohexyl (meth)acrylic, heptyl (meth)acrylic, isoheptyl (meth)acrylic, octyl (meth)acrylic, isooctyl (meth)acrylic, as well as combinations of any of the above.

The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. Additional and non-limiting examples include C1-C8 alkoxy (meth)acrylates, such as methoxy (meth)acrylate, ethoxy (meth)acrylate, propyl oxide (meth)acrylate, isopropyl oxide (meth)acrylate, butyl oxide (meth)acrylate, tert-butyl oxide (meth)acrylate, pentyl oxide (meth) acrylate, isopentyl oxide (meth)acrylate, neopentyl oxide (meth)acrylate. The esters may be, by way of non-limiting example, C2-C6 hydroxy alkyl (meth)acrylates, such as hydroxy ethyl (meth)acrylate, 2-hydroxypropyl (meth)acrylate, glycidyl (meth)acrylate, ethylene glycol di(meth)acrylate, polyethylene glycol mono(meth)acrylate, 1,4-butane diol di(meth)acrylate, 1,6,hexane diol di(meth)acrylate, and any combination thereof. The esters may be, by way of non-limiting example, aryl (meth)acrylates such as benzyl (meth)acrylate, phenyl (meth)acrylate, and any combination thereof. The esters can further contain amino groups such as aminoethyl (meth)acrylate, N,N-dimethylaminoethyl (meth)acrylate, N, N-dimethylaminopropyl (meth)acrylate, N, N-dimethylaminodimethylpropyl (meth)acrylate, N,N-diethyleaminoethyl (meth)acrylate, and N,N,N-trimethylaminoethyl (meth)acrylate; and salts of the ethylenic amines.

According to at least certain exemplary embodiments, the alkyl group of the esters may be either fluorinated or perfluorinated, e.g. some or all of the hydrogen atoms of the alkyl group are substituted with fluorine atoms. The monomers can also be fluorine-containing monomers, such as, by way of non-limiting example, trifluoroethyl methacrylate, 2,2,3,3-tetrafluoropropyl methacrylate, 2,2,3,3,4,4-hexafluorobutyl methacrylate, perfluorooctyl methacrylate and perfluorooctyl acrylate; and silicone macromonomers.

The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, α-methylstyrene, chlorostyrene, alkylstyrene, divinylbenzene and diallyl phthalate, and combination thereof. Other non-limiting ionic monomers can include para-styrensulfonic, vinylsulfonic, 2-(meth)acryloyloxyethylsulfonic, 2-(meth)acrylamido-2-methylpropylsulfonic acids.

The list of monomers given is not limiting, and it should be understood that it is possible to use any monomer known to those skilled in the art which includes acrylic and/or vinyl monomers (including monomers modified with a silicone chain).

In at least certain, non-limiting exemplary embodiments, acrylate latex polymers may be chosen from aqueous dispersions of Methacrylic Acid/Ethyl Acrylate copolymer (INCI: Acrylates Copolymer, such as LUVIFLEX® SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as FIXATE SUPERHOLD™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as DAITOSOL 5000AD, Daito Kasei Kogyo), Acrylates Copolymers, such as those known under the tradename LUVIMER® MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename ACUDYNE BOLD from Dow Chemical, Styrene/Acrylates/Ammonium Methacrylate Copolymer, known under the tradename SYNTRAN PC5620 CG from Interpolymer, and mixtures thereof.

In yet further exemplary and non-limiting embodiments, the film-forming latex polymers may be chosen from carboxyl functional polyurethane latex polymers, such as aqueous polyurethane dispersions. These polyurethanes are conventionally formed by the reaction of prepolymer (i) with a coreactant (ii) to produce a terminated or pendant carboxyl functional polyurethane polymer. The prepolymer (i) may have the structure according to the formula:

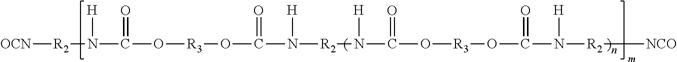

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups or potential ionic groups, in ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

The polyester diol(s) may optionally be prepared from aliphatic, cycloaliphatic, or aromatic dicarboxylic or polycarboxylic acids, or anhydrides thereof; and dihydric alcohols such as diols chosen from aliphatic, alicyclic, or aromatic diols.

The aliphatic dicarboxylic or polycarboxylic acids may be chosen from, for example: succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexanedicarboxylic, 2,5-norboranedicarboxylic, diglycolic, thiodipropionic, 2,5-naphthalenedicarboxylic, 2,6-naphthalenedicarboxylic, phthalic, terephthalic, isophthalic, oxanic, o-phthalic, tetrahydrophthalic, hexahydrophthalic or trimellitic acid.

The acid anhydrides may, in further exemplary embodiments, be chosen from o-phthalic, trimellitic or succinic acid anhydride or a mixture thereof. By way of non-limiting example only, the dicarboxylic acid may be adipic acid.

The dihydric alcohols may be chosen from, for example, ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol, and mixtures thereof. The cycloaliphatic and/or aromatic dihydroxyl compounds may also be suitable as the dihydric alcohol(s) for the preparation of the polyester polyol(s).

The polyester diols may also be chosen from homopolymers or copolymers of lactones, which are, in at least certain embodiments, obtained by addition reactions of lactones or lactone mixtures, such as butyrolactone, ε-caprolactone and/or methyl-ε-caprolactone with the appropriate polyfunctional, e.g. difunctional, starter molecules such as, for example, the dihydric alcohols mentioned above. The corresponding polymers of ε-caprolactone may be chosen in at least some embodiments.

The polyester polyol, e.g. polyester diol, radical R1, may be obtained by polycondensation of dicarboxylic acids, such as adipic acid, with polyols, e.g. diols, such as hexanediol, neopentyl glycol, and mixtures thereof.

The polycarbonates containing hydroxyl groups comprise those known per se, such as the products obtained by reacting diols, such as (1,3)-propanediol, (1,4)-butanediol and/or (1,6)-hexanediol, diethylene glycol, triethylene glycol, or tetraethylene glycol with diaryl carbonates, for example diphenyl carbonate or phosgene.

Optional polyether polyols may be obtained in any known manner by reacting starting compounds which contain reactive hydrogen atoms with alkylene oxides, such as, for example, ethylene oxide; propylene oxide; butylene oxide; styrene oxide; tetrahydrofuran; or epichlorohydrin, or with mixtures of these alkylene oxides. In at least certain embodiments, the polyethers do not contain more than about 10% by weight of ethylene oxide units. For example, polyethers obtained without addition of ethylene oxide may be chosen.

Polyethers modified with vinyl polymers are also suitable according to various embodiments of the disclosure. Products of this type can be obtained by polymerization, for example, of styrene and acrylonitrile in the presence of polyethers, for example as described in U.S. Pat. Nos. 3,383,351; 3,304,273; 3,523,095; 3,110,695; and German patent 1 152 536.

Among the polythioethers which may be chosen include the condensation products obtained from thiodiglycol per se and/or with other glycols, dicarboxylic acids, formaldehyde, aminocarboxylic acids, and/or amino alcohols. The products obtained are either mixed polythioethers, polythioether esters, or polythioether ester amides, depending on the co-components.

Optional polyacetals include but are not limited to the compounds which can be prepared from aldehydes, for example formaldehyde, and from glycols, such as diethylene glycol, triethylene glycol, ethoxylated 4,4′-(dihydroxy)diphenyldimethylmethane, and (1,6)-hexanediol. Polyacetals useful according to various non-limiting embodiments of the disclosure can also be prepared by polymerization of cyclic acetals.

Optional polyhydroxy polyesteramides and polyamines include, for example, the mainly linear condensation products obtained from saturated or unsaturated, polybasic carboxylic acids or anhydrides thereof, and from saturated or unsaturated, polyvalent amino alcohols, from diamines, or from polyamines, as well as mixtures thereof.

Optional monomers for the production of polyacrylates having hydroxyl functionality comprise acrylic acid, methacrylic acid, crotonic acid, maleic anhydride, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, glycidyl acrylate, glycidyl methacrylate, 2-isocyanatoethyl acrylate, and 2-isocyanatoethyl methacrylate.

Mixtures of dihydroxy compounds can also be chosen.

Optional polyisocyanates for providing the hydrocarbon-based radical R2 include, for example, organic diisocyanates having a molecular weight ranging from about 100 to about 1500, such as about 112 to about 1000, or about 140 to about 400.

Optional diisocyanates are those chosen from the general formula R₂(NCO)₂, in which R₂ represents a divalent aliphatic hydrocarbon group comprising from about 4 to 18 carbon atoms, a divalent cycloaliphatic hydrocarbon group comprising from about 5 to 15 carbon atoms, a divalent aromatic hydrocarbon group comprising from about 7 to 15 carbon atoms, or a divalent aromatic hydrocarbon group comprising from about 6 to 15 carbon atoms. Examples of the organic diisocyanates which may be chosen include, but are not limited to, tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis(4-isocyanatocyclohexyl)-methane, 1,3-bis(isocyanatomethyl)cyclohexane and 1,4-bis(isocyanatomethyl)cyclohexane and bis(4-isocyanato-3-methylcyclohexyl)methane. Mixtures of diisocyanates can also be used.

In at least certain embodiments, diisocyanates are chosen from aliphatic and cycloaliphatic diisocyanates. For example, 1,6-hexamethylene diisocyanate, isophorone diisocyanate, and dicyclohexylmethane diisocyanate, as well as mixtures thereof may be chosen.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylolbutanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Coreactants (ii) are compounds containing functional groups such as hydroxy or amine groups, in certain embodiments primary amine, adapted to react with isocyanate groups in preference to the carboxyl group according to the formula:

X—R4-X X=OH, NH2,

wherein R4 represents a divalent aliphatic or cycloaliphatic or aromatic hydrocarbon group, optionally substituted with ionic groups or potentially ionic groups. In various embodiments, compounds may optionally be chosen from alkylene diamines, such as hydrazine, ethylenediamine, propylenediamine, 1,4-butylenediamine and piperazine; In various embodiments, compounds may optionally be chosen from alkylene diols, such as ethylene glycol, 1,4-butanediol (1,4-BDO or BDO), 1,6-hexanediol.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Special compounds may be chosen from dimethylolbutanoic acid (DMBA), dimethylolpropionic acid (DMPA), or carboxyl functional polyester comprising excess equivalents of dicarboxylic acid reacted with lesser equivalents of glycol or carboxyl-containing caprolactone polyester diol.

R1, R2, R3, R4 can have at least one carboxyl group independently.

By way of non-limiting example, such latexes include, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as LUVISET® P.U.R, BASF), a copolymer of hexylene glycol, neopentyl glycol, adipic acid, saturated methylene diphenyldiisocyanate and dimethylolpropanoic acid monomers (INCI name: polyurethane 2), a copolymer of PPG-17, PPG-34, isophorone diisocyanate and dimethylolpropanoic acid monomers (INCI name: polyurethane 4), a copolymer of isophthalic acid, adipic acid, hexylene glycol, neopentyl glycol, dimethylolpropanoic acid, isophorone diisocyanate and bis-ethylaminoisobutyl-dimethicone monomers (INCI name: polyurethane 6), Isophorone diisocyanate, cyclohexanedimethanol, dimethylol butanoic acid, polyalkylene glycol and N-methyl diethanolamine copolymer (INCI name: polyurethane 10), Trimethylolpropane, neopentyl glycol, dimethylol propionic acid, polytetramethylene ether glycol and isocyanato methylethylbenzene copolymer (INCI name: polyurethane 12), Isophorone diisocyanate, dimethylol propionic acid, and 4,4′-isopropylidenediphenol reacted with propylene oxide, ethylene oxide and PEG/PPG-17/3 copolymer (INCI name: polyurethane 14), Isophorone diisocyanate, adipic acid, triethylene glycol and dimethylolpropionic acid copolymer (INCI name: polyurethane 15), 2-Methyl-2,4-pentanediol, polymer with 2,2-dimethyl-1,3-propanediol, hexanedioic acid, methylenedicyclohexanediisocyanate and 2,2-di(hydroxymethyl)propanoic acid, hydrolysed, tris(2-hydroxyethyl)amine salts, reaction products with 1,2-ethanediamine (INCI name: polyurethane 17), Polyurethane-27 is a complex polymer that is formed by the reaction of Polyperfluoroethoxymethoxy Difluorohydroxyethyl Ether and isophorone diisocyanate (IPDI) to form a prepolymer. The prepolymer is further reacted with the triethylamine salt of 3-hydroxy-2-(hydroxymethyl)-2-methyl-1-propionic acid (INCI name: polyurethane 27), a complex polymer formed by reacting dimethylolpropionic acid and a polyester composed of Adipic Acid, Hexylene Glycol, Neopentyl Glycol with methylene dicyclohexyldiisocyanate (SMDI) to form a prepolymer. The prepolymer is neutralized with triethylamine and then chain-extended with hydrazine (INCI name: polyurethane 33).

In some embodiments, the latex polymers of the present invention are film-forming latex polymers.

In certain other embodiments, the latex polymers of the present invention are non film-forming latex polymers.

As used herein, a film-forming polymer is meant to include a polymer that is capable, by itself or in the presence of an auxiliary film-forming agent, of forming a macroscopically continuous film that adheres to keratin materials, and in certain embodiments a cohesive film, better still, a film whose cohesion and mechanical properties are such that said film can be isolated and manipulated individually, for example, when said film is prepared by pouring onto a non-stick surface such as Teflon-coated or silicone-coated surface. In addition, as used herein, a non-film-forming polymer is meant to include a polymer which will not form a film at ambient temperature or below, or in other words, will only form a film at temperatures above ambient. For purposes of this disclosure, ambient temperature is taken as being below 40° C. such as in the range of 15° C. to 30° C.

By way of non-limiting example only, the latex polymers may be chosen from at least one carboxyl functional silicone latex polymer.

The carboxyl functional silicone latex polymer can be an organopolysiloxane comprising: (A) a compound having the following formula, Unit (A):

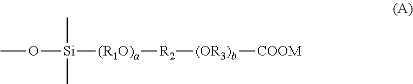

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH₄ or a quaternary ammonium group such as a mono-, di-, tri- or tetra(C₁-C₄ alkylammonium) group, and optionally substituted divalent aromatic groups, such as groups of formula (A′):

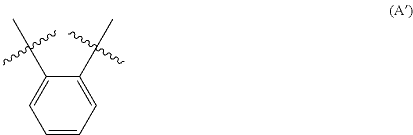 [SMILES: *c1ccccc1*]

and groups of formula (A″):

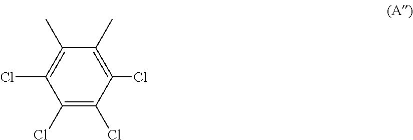

R₁ and R₃ can denote, for example, ethylene, propylene or butylene,
or

(B) a group comprising at least one pyrrolidone carboxylic acid unit having the following formula, unit (B):

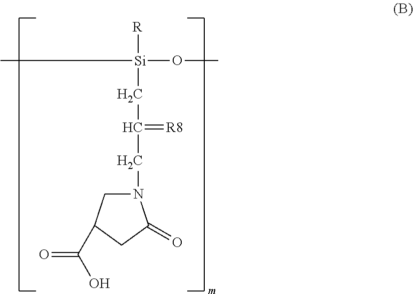

in which R is selected from methyl or phenyl; R8 is hydrogen or methyl, m is an integer from 1 to 1000, or

(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable silicone latex polymers include, for example, a silicone comprising at least one carboxylic acid group chosen from organopolysiloxanes of formula (IA):

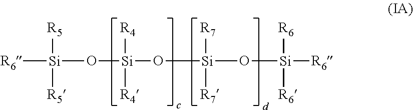

wherein the radicals R₄, R4′ are identical to or different from each other and are chosen from a linear or branched C₁-C₂₂ alkyl radical, a C₁-C₂₂ alkoxy radical and a phenyl radical, the radicals R₅, R₅′, R₅″, R₆, R₆′, R₆″, R₇, and R₇′ are identical to or different from each other and are chosen from a linear or branched C₁-C₂₂ alkyl radical, a C₁-C₂₂ alkoxy radical, a phenyl radical, a radical —(R₁O)ₐ—R₂—(OR₃)_{b}—COOM, a radical containing pyrrolidone carboxylic acid, a radical of polyvinyl acid/ester; and
wherein at least one of the radicals R₅, R₆ and R₇ is a radical chosen from a radical —(R₁O)ₐ—R₂—(OR₃)_{b}—COOM, a radical containing pyrrolidone carboxylic acid, a radical of polyvinyl acid/ester;
wherein R₁, R₂, R₃, a, b and M have the same meaning as described in Unit (A) above;
wherein c and d are integers from 0 to 1000, the sum c+d in certain embodiments ranging from 1 to 1000 or from 2 to 1000.

Among the compounds of formula (IA) that comprise at least one unit (A), the preferred ones are the compounds of formula (IIA) below:

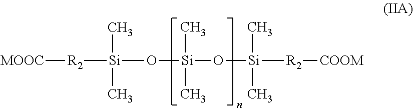

wherein R₂, and M have the same meaning as described in Unit (A) above, n is an integer from 1 to 1000.

Other preferred compounds of formula (IA) are the ones of formula (IIIA):

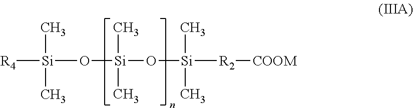

in which R₂, R₄, n, and M having the same meaning as in Unit (A) above.

Other preferred compounds of formula (IA) are the ones of formula (IVA):

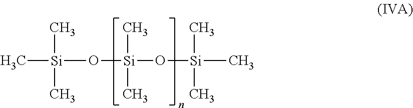

in which X is a radical —(R₁O)ₐ—R₂—(OR₃)_{b}—COOM wherein R₁, R₂, R₃, a, b and M have the same meaning as described in Unit (A) above.
Even more particularly, the compounds of formula (IVA) in which a and b are equal to 0 and R₂ is a linear or branched C₂-C₁₂ alkylene group such as (CH₂)₉, (CH₂)₁₀ or —CH(CH₃)— are preferred.

Among the compounds of formula (IA) that contain unit (B), the preferred ones are the compounds of formula (VA) below:

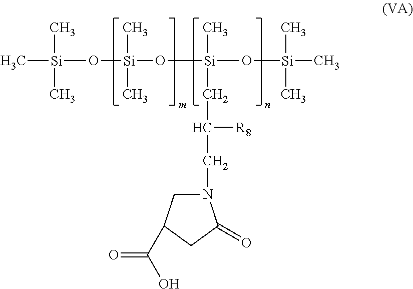

wherein R8, m, are defined as in Unit (B) above and n is an integer from 1 to 1000.

Among the organopolysiloxanes of formula (IA) that contain polyvinyl acid/ester Unit (C), the preferred ones are cross-linked anionic copolymers comprised of organic polymer blocks and silicone blocks, resulting in a multiblock polymer structure. In particular, the silicone-organic polymer compound of the present invention may be chosen from cross-linked anionic copolymers comprising at least one cross-linked polysiloxane structural unit. An example of such a branched multi-block carboxy silicone polymer is BELSIL P1101 (may also be known under the tradename BELSIL P101) (INCI name: Crotonic AcidNinyl C8-12 Isoalkyl EstersNA/Bis-Vinyldimethicone Crosspolymer, also known by the technical name of Crotonic AcidNinyl C8-12 Isoalkyl EstersNA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Solvent

The composition, according to the present disclosure, further includes suitable solvents for treatment of keratinous fibers. Examples of suitable solvents include water, in some embodiments, distilled or de-ionised, or organic solvents as carriers and solvents for the polycarbodiimides and latex polymers thereof.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, isododecane, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. An exemplary glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluoro oils such as nonafluoromethoxybutane and perfluoro methylcyclopentane.

Particularly suitable solvents in a composition of the present disclosure include water, isododecane, ethanol, and combinations thereof. The solvent will typically be present in total amounts ranging from about 60% to 98%, in some embodiments, from 80% to 96%, by weight, including all ranges and subranges therebetween, based on the total weight of the composition. Thus, solvents may be present in amounts of about 98%, 97%, 96%, 95%, 94%, 93%, 92%, 91%, 90%, 85%, 80%, 75%, 70%, 65%, 60%, and about 55%, 50%, 48%, 46%, 45%, 44%, 42%, 40%, by weight, based on the total weight of the composition, including increments and ranges therein and there between from about 40% to about 98%.

In yet some other embodiments, the solvent of the present disclosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

Additives

The composition, according to the present disclosure, further includes suitable additives for treatment of keratinous fibers. These additives are compounds other than the latex polymers of the present disclosure.

The composition according to the disclosure may also comprise additives chosen from amino compounds (e.g., amino silicones, alkyl monoamines, polyamines, alkoxylated monoamines, alkoylated polyamines, and amino functionalized silane compounds), surfactants (anionic, nonionic, cationic and amphoteric/zwtterionic), and polymers other than the polycarbodiimide compounds and latex polymers of the invention such as anionic polymers, nonionic polymers, amphoteric polymers, polymeric rheology modifiers, thickening and/or viscosity modifying agents, associative or non-associative polymeric thickeners. Other suitable additives may be chosen from non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof.

The compositions of certain embodiments may comprise stabilizers, for example sodium chloride, magnesium dichloride or magnesium sulfate.

The amine or amino compounds that may be employed in the compositions of the present disclosure are not chosen from the amino silicones of the invention. The term “amino” is intended to mean refer to a primary, secondary or tertiary amine or a quaternary ammonium group.

The amine or amino compounds that may be employed in the compositions of the present disclosure may also be chosen from alkyl monoamines, alkoxylated polyamines, alkoxylated monoamines, and polyamines, in particular, those that do not contain silicon atoms or silicone moieties.

Suitable examples of alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

The alkyl monoamines may also be chosen from amidoamines, including, but not limited to the following examples: oleamidopropyl dimethylamine, stearamidopropyl dimethylamine, isostearamidopropyl dimethylamine, stearamidoethyl dimethylamine, lauramidopropyl dimethylamine, palmitamidopropyl dimethylamine, stearamidoethyldiethylamine, and mixtures thereof.

The alkoxylated polyamines that may be employed in the compositions of the present disclosure are chosen from amino compounds having at least two amino groups and at least one degree of alkoxylation provided by an alkylene oxide group which is in certain embodiments chosen from ethylene oxide and propylene oxide.

Suitable examples of alkoxylated polyamines include, but are not limited to diamine and triamine compounds belonging to the JEFFAMINE series such as the JEFFAMINE D, JEFFAMINE ED, JEFFAMINE EDR, and JEFFAMINE T series available from Huntsman Corporation.

The alkoxylated monoamines that may be employed in the compositions of the present disclosure are chosen from amino compounds having at one amino groups and at least one degree of alkoxylation provided by an alkylene oxide group which is in certain embodiments chosen from ethylene oxide and propylene oxide.

Suitable examples are alkoxylated derivatives of cocamine, lauramine, palmitamine, rapeseedamine, oleamine, soyamine, stearamine, tallow amine, tallow aminopropylamine, behenyl propylenediamine and those of the JEFFAMINE M series from Huntsman.

The polyamines that may be used in the compositions of the present disclosure may in particular be chosen from aminosilicones, polyvinylamines, aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof.

The amino silicones that may be employed in the compositions of the present disclosure may be chosen from polysiloxanes having at least one primary, secondary or tertiary amine group such as trimethylsilylamodimethicones, quaternary ammonium silicones, multiblock polyoxyalkylenated amino silicones, of type (AB)n, A being a polysiloxane block and B being a polyoxyalkylenated block containing at least one amine group, alkyl amino silicones, and mixtures thereof.

Suitable examples of the amine or amino silicone compounds include amodimethicone (e.g., sold under the name KF 8020 by Shin Etsu or XIAMETER® MEM-8299 Cationic Emulsion by Dow Corning), and bis-cetearyl amodimethicone (sold under the name SILSOFT AX by Momentive).

The rheology modifiers and thickening/viscosity-modifying agents that may be employed in compositions of the present disclosure may include any water-soluble or water-dispersible compound that is compatible with the polycarbodiimide, amino compounds, and compositions of the disclosure, such as acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose), non-polymeric and polymeric gelling agents, silica particles, clay, and mixtures thereof.

Other Additives

Polycarboxylic acid Polymer Compounds

The polycarboxylic acid, compounds of the present disclosure may be chosen from anionic, nonionic, and amphoteric polymers.

The anionic polymers may be soluble in a cosmetically acceptable medium or insoluble in this same medium such that they may be used in the form of dispersions of solid or liquid particles of polymer (latex or pseudolatex).

The anionic polymers may be selected from polymers comprising groups derived from carboxylic acids, and have an average molecular weight by number of between about 500 and 5,000,000. The carboxylic groups are provided by unsaturated mono- or diacid carboxylic monomers such as those that have the formula:

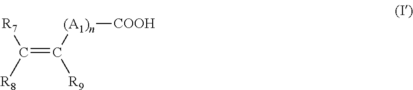

in which n is an integer from 0 to 10, A₁ denotes a methylene group optionally joined to the carbon atom of the unsaturated group or to the adjacent methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group, a CH₂—COOH, phenyl or benzyl group.

In the abovementioned formula, a lower alkyl group in certain embodiments denotes a group containing 1 to 4 carbon atoms and in particular methyl and ethyl groups. Examples of anionic polymers containing carboxyl groups in accordance with the invention are:

The polycarboxylic acid compounds include Copolymers of acrylic or methacrylic acid or salts thereof, and in particular copolymers of acrylic acid and acrylamide sold in the form of their sodium salts;

The polycarboxylic acid compounds include Copolymers of acrylic or methacrylic acid with a monoethylenic monomer such as ethylene, styrene, vinyl esters and acrylic or methacrylic acid esters, optionally grafted onto a polyalkylene glycol such as polyethylene glycol and optionally crosslinked. The polycarboxylic acid compounds include methacrylic acid/acrylic acid/ethyl acrylate/methyl methacrylate copolymers in an aqueous dispersion.

The polycarboxylic acid compounds include Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an [alpha]- or [beta]-cyclic carboxylic acid.

The polycarboxylic acid compounds include Copolymers of C4-C8 monounsaturated carboxylic acids selected from: copolymers comprising (i) one or more maleic, fumaric, itaconic, allyloxyacetic, methallyloxyacetic, 3-allyloxypropionic, allylthioacetic, allylaminoacetic, vinylacetic, vinyloxyacetic, crotyloxyacetic, 3-butenoic, 4-pentenoic, 10-undecenoic, allylmalonic, maleamic, itaconamic, N-monohydroxyalkyl- or N-dihydroxy-alkyl-maleamic acids and (ii) at least one monomer selected from vinyl esters, vinyl ethers, vinyl halides, phenylvinyl derivatives, acrylic acid and its esters, the anhydride functions of these copolymers optionally being monoesterified or monoamidated. The polycarboxylic acid compounds include copolymers comprising (i) one or more maleic, citraconic or itaconic anhydride units and (ii) one or more monomers selected from allyl or methallyl esters optionally comprising one or more acrylamide, methacrylamide, [alpha]-olefin, acrylic or methacrylic ester, acrylic or methacrylic acid or vinylpyrrolidone groups in their chain, the anhydride functions of these copolymers optionally being monoesterified or monoamidated. The polycarboxylic acid compounds include polyacrylamides comprising carboxylate groups.

The polycarboxylic acid compounds of the present disclosure may also include those anionic polymers as sold under the FIXATE series as commercially available from Lubrizol, such as a branched block anionic polymer sold as FIXATE G-100, a branched anionic acrylate copolymer Polyacrylate-2 Crosspolymer (FIXATE SUPERHOLD polymer), Acrylates Crosspolymer-3 (FIXATE FREESTYLE Polymer), Polyacrylate-14 (FIXATE PLUS Polymer), those sold under the CARBOPOL series as commercially available from Lubrizol such as Acrylates Crosspolymer-4 (CARBOPOL AQUA SF-2), Acrylates Crosspolymer-4 (CARBOPOL AQUA CC), and those sold under the SYNTRAN series as commercially available from Interpolymer such as Acrylates Copolymer (SYNTRAN 5190), Styrene/Acrylates/Ammonium Methacrylate Copolymer (SYNTRAN 5760), and Ammonium Acrylates Copolymer (SYNTRAN KL-219C).

The polycarboxylic acid compounds of the present disclosure also includes anionic latex polymers such as acrylic copolymer and (meth)acrylate copolymers dispersions.

The polycarboxylic acid compounds include copolymers of acrylic acid or of acrylic esters, such as Acrylates/t-Butylacrylamide copolymer sold as ULTRAHOLD 8, acrylic acid/ethyl acrylate/N-tert-butylacrylamide terpolymers sold especially as ULTRAHOLD STRONG by BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially as RESYN 28-29-30 by Azko Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, as GANTREZ AN or ES by ISP, the copolymers of methacrylic acid and methyl methacrylate sold as EUDRAGIT L by Rohm Pharma, the copolymers of methacrylic acid and ethyl acrylate sold as LUVIMER MAEX or MAE by BASF, the vinyl acetate/crotonic acid copolymers sold as LUVISET CA 66 by BASF, the vinyl acetate/crotonic acid copolymers grafted with polyethylene glycol sold as ARISTOFLEX A by BASF, and the polymer sold as FIXATE G-100 by Noveon.

The polycarboxylic acid compounds include amphoteric polymers which may be selected from the following polymers:

copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537.

The polycarboxylic acid compounds include Polymers comprising units derived from:

at least one monomer selected from acrylamides and methacrylamides substituted on the nitrogen atom with an alkyl group,

at least one acidic comonomer containing one or more reactive carboxylic groups, selected more particularly from acrylic, methacrylic, crotonic, itaconic, maleic and fumaric acid and alkyl monoesters, having 1 to 4 carbon atoms, of maleic or fumaric acid or anhydride, and

at least one basic comonomer such as esters with primary, secondary, tertiary or quaternary amine substituents of acrylic and methacrylic acids and the product of quaternization of dimethylaminoethyl methacrylate with dimethyl or diethyl sulfate. The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides. The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold as AMPHOMER LV 71, Acrylates/octylacrylamide copolymer sold as Amphomer 28-4961 or LOVOCRYL 47 by National Starch, are particularly used.

The polycarboxylic acid compounds include Crosslinked and acylated polyaminoamides

The polycarboxylic acid compounds include carboxylic acids selected from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The polycarboxylic acid compounds include Polymers comprising zwitterionic units of formula:

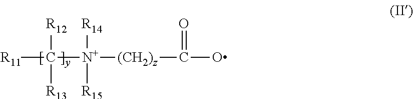

in which R11 denotes a polymerizable unsaturated group such as an acrylate, methacrylate, acrylamide or methacrylamide group, y and z represent an integer from 1 to 3, R12 and R13 represent a hydrogen atom, a methyl, ethyl or propyl group, R14 and R15 represent a hydrogen atom or an alkyl group such that the sum of the carbon atoms in R14 and R15 does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate, for example, methyl methacrylate/methyl dimethylcarboxymethylammonioethyl methacrylate copolymers such sold as DIAFORMER Z301 by Sandoz; The polycarboxylic acid compounds include Polymers derived from chitosan comprising monomer units corresponding to the following formulae:

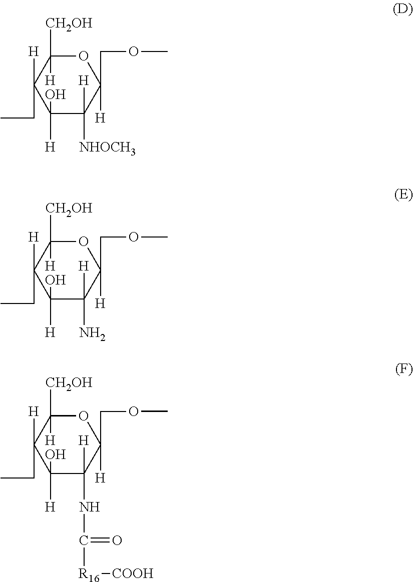

the unit (D) being present in proportions of between 0 and 30 percent, the unit (E) in proportions of between 5 percent and 50 percent and the unit (F) in proportions of between 30 percent and 90 percent, it being understood that, in this unit (F), R16 represents a group of formula:

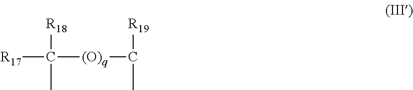

in which, if q=0, R17, R18 and R19, which may be identical or different, each represent a hydrogen atom, a methyl, hydroxyl, acetoxy or amino residue, a monoalkylamine residue or a dialkylamine residue that are optionally interspersed with one or more nitrogen atoms and/or optionally substituted with one or more amine, hydroxyl, carboxyl, alkylthio or sulfonic groups, an alkylthio residue in which the alkyl group bears an amino residue, at least one of the groups R17, R18 and R19 being, in this case, a hydrogen atom;

or, if q=1, R17, R18 and R19 each represent a hydrogen atom, and also the salts formed by these compounds with bases or acids.

The polycarboxylic acid compounds include Polymers with units corresponding to the general formula below are described, for example, in French patent 1 400 366:

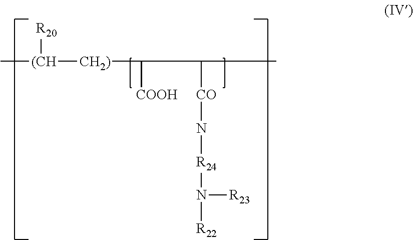

in which R20 represents a hydrogen atom, a CH3O, CH3CH2O or phenyl group, R21 denotes a hydrogen atom or a lower alkyl group such as methyl or ethyl, R22 denotes a hydrogen atom or a C1-C6 lower alkyl group such as methyl or ethyl, R23 denotes a C1-C6 lower alkyl group such as methyl or ethyl or a group corresponding to the formula: —R24-N(R22)2, R24 representing a group —CH2-CH2-, —CH2-CH2-CH2- or —CH2-CH(CH3)-, R22 having the meanings mentioned above.

The polycarboxylic acid compounds include Polymers derived from the N-carboxyalkylation of chitosan, such as N-carboxymethylchitosan or N-carboxybutylchitosan.

The polycarboxylic acid compounds include Amphoteric polymers of the type -D-X-D-X selected from:

a) Polymers obtained by the action of chloroacetic acid or sodium chloroacetate on compounds comprising at least one unit of formula:

-D-X-D-X-D- (V′)

where D denotes a

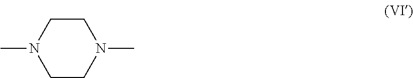

group and X denotes the symbol E or E′; E or E′ may be identical or different and denote a divalent group that is an alkylene group with a straight or branched chain containing up to 7 carbon atoms in the main chain, which is unsubstituted or substituted by hydroxyl groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) Polymers having the formula:

-D-X-D-X— (VI′)

where D denotes a

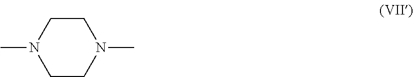

group and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interspersed by an oxygen atom and necessarily comprising one or more carboxyl functions or one or more hydroxyl functions and betainized by reaction with chloroacetic acid or sodium chloroacetate.

The polycarboxylic acid compounds include (C1-C5)Alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

Among the amphoteric polymers described above, the ones that are most preferred are Octylacrylamide/acrylates/butylamino ethyl methacrylate copolymer, such as the products sold as AMPHOMER, AMPHOMER LV 71 or LOVOCRYL 47 by National Starch and the copolymers of methyl methacrylate/methyl dimethylcarboxy-methylammonioethyl methacrylate, sold, for example, as DIAFORMER Z301 by Sandoz.

The polycarboxylic acids of the present disclosure may be chosen from compounds which are known to be used as rheology modifiers or thickeners in cosmetic compositions. Such polymers include anionic and amphoteric polymers, for example crosslinked homopolymers of acrylic acid, associative polymers, non-associative thickening polymers, and water-soluble thickening polymers. Such polymers may also be chosen from nonionic, anionic, cationic and amphoteric amphiphilic polymers. The rheology modifiers or thickeners that can used may include those polycarboxylic acid compounds described above.

The amphiphilic polymers may, optionally, contain a hydrophobic chain that is a saturated or unsaturated, aromatic or non-aromatic, linear or branched C6-C30 hydrocarbon-based chain, optionally comprising one or more oxyalkylene (oxyethylene and/or oxypropylene) units.

Representative examples of such amphiphilic polymers are:

nonionic amphiphilic polymers containing a hydrophobic chain such as:

copolymers of C1-C6 alkyl(meth)acrylates and of amphiphilic monomers containing a hydrophobic chain;

copolymers of hydrophilic (meth)acrylates and of hydrophobic monomers containing at least one hydrophobic chain, for instance the polyethylene glycol methacrylate/lauryl methacrylate copolymer;

anionic amphiphilic polymers containing at least one hydrophobic chain which are crosslinked or non-crosslinked, contain at least one hydrophilic unit derived from one or more ethylenically unsaturated monomers bearing a carboxylic acid function, which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Examples of anionic amphiphilic polymers include CARBOPOL ETD-2020 (acrylic acid/C10-C30 alkyl methacrylate crosslinked copolymer sold by the company Noveon); CARBOPOL 1382, PEMULEN TR1 and PEMULEN TR2 (acrylic acid/C10-C30 alkyl acrylatecrosslinked copolymers-sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (ACULYN 28 sold by Rohm and Haas) and the methacrylic acid/ethyl acrylate/steareth-10 allyl ether crosslinked copolymer.

Other examples include cross-linked acrylic polymers, for example those sold under the CARBOPOL SF series, such as ethyl acrylate/methacrylic acid copolymer with INCI name: acrylates copolymer, sold under the name CARBOPOL SF1(R) by the LUBRIZOL company.

Yet other examples include anionic polymers also known as anionic thickening polymers chosen from carbomers, acrylate copolymers, and crosslinked terpolymers of methacrylic acid, ethylacrylate, and polyethylene glycol (10 EO) stearyl alcohol ether (Steareth 10), such as the products sold by the company ALLIED COLLOIDS under the names SALCARE SC 80 and SALCARE SC 90, which are aqueous emulsions containing 30 percent of a crosslinked terpolymer of methacrylic acid, of ethyl acrylate and of steareth-10-allyl ether (40/50/10).

Anionic thickening polymers can also be chosen from:

terpolymers formed from maleic anhydride/C30-C38 alpha-olefin/alkyl maleate such as the product (maleic anhydride/C30-C38 alpha-olefin/isopropyl maleate copolymer) sold under the name PERFORMA 1608 by the company NEWPHASE TECHNOLOGIES;

acrylic terpolymers formed from: (a) 20 percent to 70 percent by weight of a carboxylic acid with alpha, beta-monoethylenic unsaturation; (b) 20 percent to 80 percent by weight of a nonsurfactant monomer with alpha, beta-monoethylenic unsaturation different from (a); and (c) 0.5 percent to 60 percent by weight of a nonionic monourethane which is the product of the reaction of a monohydric surfactant with a monoisocyanate with monoethylenic unsaturation; (3) copolymers formed from at least two monomers, wherein at least one of the two monomers is chosen from a carboxylic acid with alpha, beta-monoethylenic unsaturation, an ester of a carboxylic acid with alpha, beta-monoethylenic unsaturation, and an oxyalkylenated fatty alcohol; and (4) copolymers formed from at least three monomers, wherein at least one of the three monomers is chosen from a carboxylic acid with alpha, beta-monoethylenic unsaturation, at least one of the three monomers is chosen from an ester of a carboxylic acid with alpha beta-monoethylenic unsaturation and at least one of the three monomers is chosen from an oxyalkylenated fatty alcohol.

Additionally, these compounds can also contain, as a monomer, a carboxylic acid ester comprising an alpha, beta-monoethylenic unsaturation and a C1-C4 alcohol. By way of example of this type of compound, there may be mentioned ACULYN 22 sold by the company ROHM and HAAS, which is an oxyalkylenated stearyl methacrylate/ethylacrylate/methacrylic acid terpolymer.

The polycarboxylic acid compounds include associative polyurethanes, associative unsaturated polyacids, and associative polymers or copolymers containing at least one monomer comprising ethylenic unsaturation.

A representative example of an associative polyurethane is methacrylic acid/methyl acrylate/ethoxylated (40 EO) behenyl alcohol dimethyl(meta-isopropenyl)benzyl isocyanate terpolymer as a 25 percent aqueous dispersion, known by the trade name, VISCOPHOBE DB 1000 and commercially available from Amerchol.

According to some embodiments of the present invention, the polycarboxylic acid compounds include at least one acrylic acid-based, (meth)acrylic acid-based, acrylate-based or (meth)acrylate-based monomer having anionic and/or cationic functionalities. Suitable compounds include, but are not limited to, polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities. According to certain embodiments, the bimodal agent is selected from the group consisting of polymers consisting of polyacrylate-21 and acrylates/dimethylaminoethylmethacrylate copolymer (marketed under the name SYNTRAN PC 5100 by Interpolymer), polyacrylate-16 (marketed under the name SYNTRAN PC 5112 by Interpolymer), and polyacrylate-18 and polyacrylate-19 (marketed under the names SYNTRAN PC 5107 or SYNTRAN PC 5117 by Interpolymer).

Silicone Polymers Having at Least One Carboxylic Acid Group (Carboxysilicone Polymers).

The silicone polymers having at least one carboxylic acid group, referred herein as carboxysilicone polymers, according to the present disclosure, may be an organopolysiloxane comprising:

(A) a compound having the following formula:

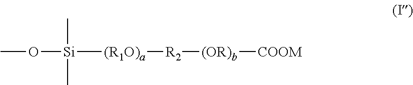

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or

(B) a group comprising at least one pyrrolidone carboxylic acid unit having the following formula:

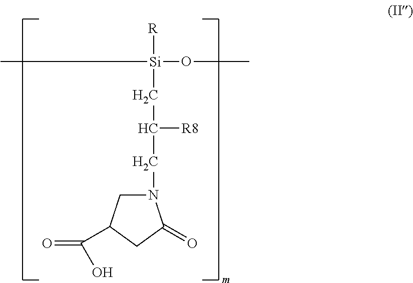

in which R is selected from methyl or phenyl; R8 is hydrogen or methyl, m is an integer from 1 to 1000, or

(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable carboxysilicone polymers include, for example, a silicone polymer comprising at least one carboxylic acid group chosen from organopolysiloxanes of formula:

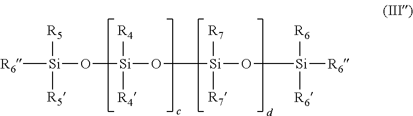

wherein the radicals R4, R4′ are identical to or different from each other and are chosen from a linear or branched C1-C22 alkyl radical, a C1-C22 alkoxy radical and a phenyl radical, the radicals R5, R5′, R5″, R6, R6′, R6″, R7, and R7′ are identical to or different from each other and are chosen from a linear or branched C1-C22 alkyl radical, a C1-C22 alkoxy radical, a phenyl radical, a radical —(R1O)a-R2-(OR3)b-COOM, a radical containing pyrrolidone carboxylic acid, a radical of polyvinyl acid/ester; and

wherein at least one of the radicals R5, R6 and R7 is a radical chosen from a radical —(R10O)a-R2-(OR3)b-COOM, a radical containing pyrrolidone carboxylic acid, a radical of polyvinyl acid/ester;

wherein R1, R2, R3, a, b and M have the same meaning as described in Unit (A) above;

wherein c and d are integers from 0 to 1000, the sum c+d in some particular embodiments ranging from 1 to 1000 or from 2 to 1000.

Among the carboxysilicone polymers of formula (III″) that comprise at least one unit (I″), which in some particular embodiments are the compounds of formula below:

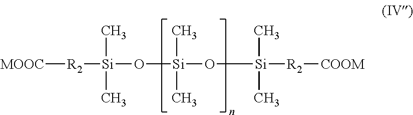

wherein R2, and M have the same meaning as described in Unit (A) above, n is an integer from 1 to 1000. Examples are: dual-end carboxy silicones X-22-162C from Shin Etsu and Silform INX (INCI name: Bis-Carboxydecyl Dimethicone) from Momentive.

Other exemplary embodiments organopolysiloxanes of formula (III″) are the ones of formula:

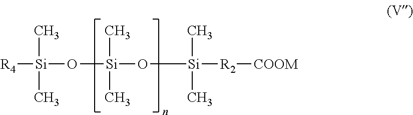

in which R₂, R₄, n, and M having the same meaning as in Unit (A) above. An example is a single-end carboxy silicone X-22-3710 from Shin Etsu.

Other exemplary embodiments organopolysiloxanes of formula (III″) are the ones of formula:

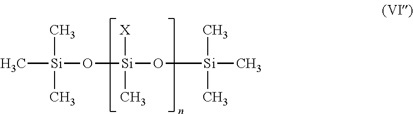

wherein X is a radical —(R1O)a-R2-(OR3)b-COOM wherein R1, R2, R3, a, b and M have the same meaning as described in Unit (A) above.

Even more particularly, the compounds of formula (VI′) in which a and b are equal to 0 and R₂ is a linear or branched C₂-C₁₂ alkylene group such as (CH₂)₉, (CH₂)₁₀ or —CH(CH₃)— are exemplary embodiments. An example is a side-chain carboxy silicone X-22-3701E from Shin Etsu.

Among the organopolysiloxanes of formula (III″) that contain unit (B), exemplary embodiments include the compounds of formula below:

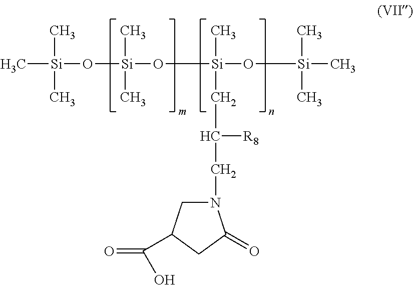

wherein R8, m, are defined as in Unit (B) above and n is an integer from 1 to 1000. An example is Grandsil PCA such as in Grandsil SiW-PCA-10 (INCI name: Dimethicone (and) PCA Dimethicone (and) Butylene Glycol (and) Decyl Glucoside from Grant Industries.

Among the organopolysiloxanes of formula (III″) that contain polyvinyl acid/ester Unit (C), exemplary embodiments are crosslinked anionic copolymers comprised of organic polymer blocks and silicone blocks, resulting in a multiblock polymer structure. In particular, the silicone-organic polymer compound of the present invention may be chosen from crosslinked anionic copolymers comprising at least one crosslinked polysiloxane structural unit. An example of such a branched multi-block carboxysilicone polymer is Belsil® P1101 (may also be known under the tradename Belsil® P1101) (INCI name: Crotonic Acid/Vinyl C8-12 Isoalkyl EstersNA/Bis-Vinyldimethicone Crosspolymer, also known by the technical name of Crotonic AcidNinyl C8-12 Isoalkyl EstersNA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Compounds corresponding to formula (VI″) above are sold, for example, under the name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone.

Other non-limiting examples of carboxysilicone polymers are silicone carboxylate containing polymers (silicone carboxylates).

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the silicone carboxylate is a compound of formula:

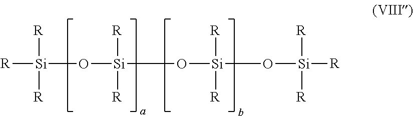

wherein a is an integer ranging from 1 to 100; b is an integer ranging from 0 to 500; and R, which may be identical or different, are each chosen from optionally substituted hydrocarbon groups comprising from 1 to 9 carbon atoms, optionally substituted phenyl groups, and groups of the following formula:

—(CH₂)₃—O-(EO)_{c}—(PO)_{d}-(EO)ₑ—C(O)—R′—C(O)—OH (IX″)

wherein c, d, and e, which may be identical or different, are each integers ranging from 0 to 20; EO is an ethylene oxide group; PO is a propylene oxide group; and R′ is chosen from optionally substituted divalent hydrocarbons, such as alkylene groups and alkenylene groups comprising from 2 to 22 carbon atoms, and optionally substituted divalent aromatic groups, such as groups of the following formula:

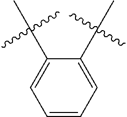 [SMILES: *c1ccccc1*]

and groups of the following formula:

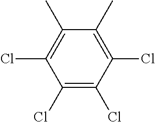

with the proviso that at least one of the R groups is chosen from groups of formula (XIV) and with the further proviso that when only one of the R groups is chosen from groups of formula (XIV), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (XV) below. Thus, in one embodiment, the at least one silicone carboxylate is chosen from a compound of formula below and salts thereof:

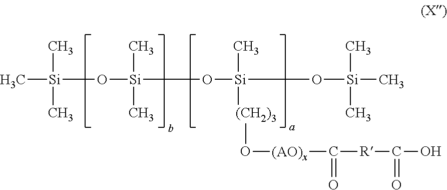

wherein a is an integer ranging from 1 to 100, b is an integer ranging from 0 to 500, AO is chosen from groups of the following formula:

-(EO)c-(PO)d-(EO)e-

wherein c, d, and e, which may be identical or different, are each integers ranging from 0 to 20; EO is an ethylene oxide group; PO is a propylene oxide group; x is an integer ranging from 0 to 60; R′ is chosen from optionally substituted divalent hydrocarbons, such as alkylene groups and alkenylene groups comprising from 2 to 22 carbon atoms, and optionally substituted divalent aromatic groups, such as groups of the following formula:

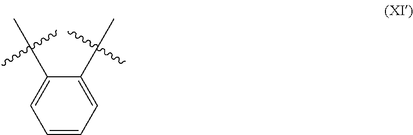 [SMILES: *c1ccccc1*]

and groups of formula

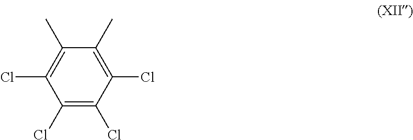

Non-limiting examples of silicone carboxylates include those described in U.S. Pat. Nos. 5,248,783 and 5,739,371, the disclosures of which are incorporated herein by reference, and which are silicone compounds of formula (XII″).

Cationic Polymers Containing at Least One Carboxyl Group.

The cationic polymer can have a negative charge but remains cationic overall, can be an amphoteric polymer that can carry a cationic charge based on pH, or can be a betaine polymer that remains amphoteric at any pH.

The cationic polymers are polymers that result from the homopolymerization or copolymerization of ethylenically unsaturated monomers chosen from: (i) at least one nonionic monomer such as (Alkyl)(Meth)Acrylamide, (Alkyl)(Meth)Acrylate Ester, Vinyl Pyrrolidone, Vinyl Imidazole; (ii) at least one cationic monomer such as Ethyltrimonium (Alkyl)(Meth)Acrylamide, Ethyltrimonium (Alkyl)(Meth)Acrylate Ester, Vinylimidazoline, Dimethylaminopropyl (Alkyl)(Meth)Acrylamide, Methacrylamidopropyl Triethyl Ammonium Chloride (MAPTAC), Diallyl Dimethyl Ammonium Chloride (DADMAC); (iii) at least one (Alkyl)Acrylic acid; (iv) at least one amphoteric monomer such as a carboxybetaine zwitterionic monomer.

Suitable examples of such cationic polymers are: the diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53); Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof.

Carboxylic acid compound chosen from fatty acids, their salts, and mixtures thereof.

The carboxylic acid compound may generally be chosen from saturated or unsaturated carboxylic acids having carbon chains containing from 6 to 30 carbon atoms, in certain embodiments from 9 to 30 carbon atoms, and in certain embodiments from 9 to 22 carbon atoms and wherein the carbon chain is optionally substituted, for example with one or more (in particular 1 to 4) hydroxyl groups. If the fatty acids of the present disclosure are unsaturated, these compounds may comprise one to three conjugated or unconjugated carbon-carbon double bonds.

Suitable examples of the carboxylic acid compound of the present disclosure are oleic acid, linoleic acid, linolenic acid, isostearic acid, caproic acid, capric acid, caprylic acid, oleic acid, linoleic acid, linolenic acid, behenic acid, lauric acid, myristic acid, stearic acid, palmitic acid and mixtures thereof.

The carboxylic acid compound of the present disclosure may also be chosen from salts of fatty acids, in particular, alkali metal salts of fatty acids (metal soaps) and organic base salts of fatty acids.

The metal of the alkali metal salts of fatty acids includes sodium, potassium, lithium and their mixtures. The organic base salts of fatty acids may be obtained from the neutralization of fatty acids with organic bases such as ammonia, monoethanolamine or triethanolamine. Suitable examples include sodium stearate, zinc laurate, magnesium stearate, magnesium myristate, zinc stearate, potassium cocoate ammonium stearate, ammonium oleate, ammonium nonanoate, and their mixtures.

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods of Preparation and Methods of Use

In some embodiments, the compositions of the present disclosure are prepared by combining the polycarbodiimide, latex polymer, and a solvent.

In other embodiments, the compositions of the present disclosure are prepared by combining the polycarbodiimide, latex polymer, and a solvent chosen from water, organic solvents, and mixtures thereof.

In yet other embodiments, the compositions of the present disclosure are prepared by combining the polycarbodiimide, latex polymer, and a solvent comprising water and organic solvents.

One-Step or Multi-Step Application Processes

In various embodiments, treatment regimens for keratinous substrates may include single or sequential applications of compositions comprising one or more of a polycarbodiimide and a latex polymer. According to the various one and multi-step embodiments, the compositions of each mixture or phase may be applied once or more than once.

One-Step Application Process

In one embodiment, the composition of the present disclosure is applied onto keratinous substrates such as hair in a one-step application process. The composition for use in the one-step application is prepared by combining the ingredients, including the polycarbodiimide, latex polymer, and solvent, resulting in a composition that is then applied onto the keratinous substrate.

In one embodiment, when the composition for a one-step application process comprises a solvent comprising water and an organic solvent, the polycarbodiimide is combined with water to form on an aqueous phase and the latex polymer is combined with an organic solvent to form a non-aqueous phase. Both phases are then combined and agitated to form an emulsion. The formed emulsion is then applied to a keratinous substrate such as hair for treatment.

Two-Step Application Process

In other embodiments, the composition of the present disclosure is applied onto keratinous substrates such as hair in a two-step application process. In a two-step application process, the individual components (polycarbodiimide combined with a solvent and latex polymer combined with a solvent) are applied to the keratinous substrate in a step-wise fashion in any order to treat the substrate.

In one such embodiment, the keratinous substrate is first treated with the polycarbodiimide phase, followed by treatment with the latex polymer phase.

In another embodiment, referred to herein as a reverse-step system, compositions comprising at least one polycarbodiimide and at least one latex polymer are prepared as separate phases, and each phase is applied separately to the keratinous substrate, such as hair. In one such embodiment, the keratinous substrate is first treated with the latex polymer phase, followed by treatment with the polycarbodiimide phase.

Three-Step Application Process

In other embodiments, the composition of the present disclosure is applied onto keratinous substrates such as hair in a three-step application process. In a three-step application process, the individual components (polycarbodiimide combined with a solvent and latex polymer combined with a solvent) are applied to the keratinous substrate in a step-wise fashion in any order to treat the substrate.

In one such embodiment, the keratinous substrate is first treated with the latex polymer phase, followed by treatment with the polycarbodiimide phase, followed by treatment with the latex polymer phase.

The application of the composition onto a keratinous substrate such as hair, according to the present disclosure, may occur at room temperature.

In other embodiments, the application of the composition according to the present disclosure, may occur at an elevated temperature (or temperatures greater than room temperature) by applying heat to the hair or exposing hair to elevated temperatures. While not so limited, heating may be provided, for example, by commonly used heating tools for example a helmet dryer or blow dryer (40° C. and above) or hot iron or flat iron (120° C.-250° C.) or steam/hot rollers.

The composition and treatment, according to the present disclosure, provides advantageous properties to keratinous fibers. In one embodiment, the composition and treatment, according to the present disclosure, provides hydrophobicity or imparts hydrophobicity to hydrophilic or damaged keratinous fibers, such as damaged hair (platinum bleached), upon application thereto. In certain embodiments, hydrophobicity is provided at room temperature, i.e., without heating or applying heat to the hair. In other embodiments, the hydrophobicity is provided when heat is used on the hair (before or after applying the composition on the hair or during the application of the composition on the hair).

In one embodiment, the hydrophobicity provided to less hydrophobic or hydrophilic keratinous fibers includes a contact angle of greater than 50° or greater than 70° or greater than 90°, or greater than 100° or ranging from between about 50° and about 120°, such as from between about 50° and about 1100.

For example, hair treated with the inventive compositions, when heated at 50° C. for 30 minutes and then allowed to cool down for a period of time at room temperature, exhibited increased percent curl retention indicating higher resistance to high humidity and high temperature compared to hair treated with either material alone. With the use of heat (drying in the oven), the percent curl retained using the inventive composition was greater than that obtained when heat was not applied or employed on hair. Accordingly, the composition, according to the present disclosure, provides increased curling benefits when utilized, particularly when the keratinous fibers are exposed to commonly used heating tools, such as a blow dryer (60° C.) or hot iron or flat iron (120° C.-250° C.) or a heat lamp, a heat wand, or other similar devices.

The method or process of using the compositions of the present invention may depend on the type of hair being targeted and, consequently, on the specific ingredients contained in the composition used to style or shape or maintain the shape of hair.

An embodiment of the present invention is a method of styling or shaping or maintaining the shape of hair.

Another embodiment of the present invention is a method of imparting durable or long-lasting style or shape to hair comprising applying onto the hair, any one of the compositions of the present disclosure.

According to at least one embodiment, such a method comprises applying to the hair, an effective amount of any one of the compositions of the present disclosure.

The compositions of the present disclosure may be employed in an effective amount to adequately cover the surface of the fibers of the hair and to achieve a desirable or effective style or shape of the hair as well as a desirable degree of hold. The precise amount of composition to be applied onto the hair will thus depend on the degree of treatment/styling/shaping/hold desired.

An effective amount of the composition is typically from about 0.1 gram to about 50 grams, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous material, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

Further disclosed herein is the use of the compositions of the present disclosure for shaping or styling hair and/or retaining a hairstyle. Also disclosed is the use of the compositions of the present disclosure for caring for the hair such as for hair repair treatments, or for reducing damage to the hair or for improving the feel of the hair by imparting hydrophobicity to the hair.

The compositions may be applied to wet or dry hair, before or after shaping. They may be used in a non-rinse fashion. In some other embodiments, the composition may be rinsed from the hair.

The hair that has been contacted with the compositions of the present invention may be air-dried and/or further styled or shaped by applying heat on the hair and/or by combing or brushing or running the fingers through the hair. Other shaping tools may be chosen from combs and brushes.

In certain embodiments, the composition is allowed to remain (leave-on time) on the keratin fibers, for example, from about 1 to about 60 minutes, or such as from about 5 to about 45 minutes, or such as from about 5 to about 30 minutes, or such as from about 10 to about 20 minutes, or such as at about 20 minutes, or such as at about 10 minutes.

The smoothing action may be accomplished by use of suitable devices for brushing or smoothing the hair include a hair brush, comb, or flat iron. The smoothing action on the hair may also include running the fingers through the hair.

A suitable applicator device is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

Heat (at a temperature of at least 40° C.) can be applied to the hair while the smoothing action is performed on the hair. The heat source can be chosen from a blow dryer, a flat iron, a hair dryer, a heat lamp, a heat wand, or other similar devices.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes. In alternate embodiments, the treatment times may be longer, and in some embodiments, appreciably longer, such that the application may be left on for up to 24 hours to about 48 hours.

The compositions of the present invention are easy to spread on hair.

It has been surprisingly and unexpectedly discovered that the application of the composition onto the hair results in the retention of the shape or style or curl of hair or of making the hair humidity resistant, including under conditions of high humidity. It was also surprisingly and unexpectedly discovered that the application of the composition onto the hair results in improving the quality of the hair, for example, better hair feel and appearance.

The shape/styling control, the curl retention, humidity resistant, and hair care effects obtained using the compositions and methods of the present invention may also be durable or long-lasting, i.e., wash or shampoo resistant.

As used herein, “long-lasting” or “durable” is understood to mean that the benefits imparted to hair by the compositions of the invention last over a period of time and/or over high humidity conditions and/or after one or multiple wash cycles (with water or shampoo/water or shampoo/water/conditioner/water or conditioner/water). Said multiple wash cycles is understood to mean more than one wash cycle, such as two or three or four or five or six or seven or eight or nine or ten wash cycles.

Another embodiment of the present invention is method for imparting durable or long-lasting style/shape and/or curl and/or care to hair comprising (a) providing the composition of the present invention, and (b) providing instructions for applying said composition to the hair.

In at least certain exemplary embodiments, the compositions are in the form of hair styling compositions, in any form, such as, for example, a gel, a cream, a foam, a lotion, an emulsion, or a liquid that may be sprayed onto or otherwise applied to the hair. In various embodiments, the composition may be provided in the form of a gel, a mousse, or a spray. In at least certain embodiments, the composition may be applied to the hair by first applying to the hands, and then contacting the hair with the hands; in other embodiments, the composition may be applied directly onto the hair, such as by spraying. The compositions may, in various embodiments, be applied to the hair as a leave-on treatment.

In various embodiments, the application of an external stimuli, such as heat, may be desirable as part of the hair styling process. By way of example only, before, during, or after the composition is applied to wet or dry hair, the hair may optionally be further treated with an external stimuli, for example with heat ranging from about 25° C. to about 250° C. In at least certain embodiments, the hair may also be shaped or positioned as desired while exposed to external stimuli, such as while heated or exposed to heat.

Professional and consumer heating tools such as those described above can be used as a means to deliver heat or an elevated temperature to the hair.

As described, compositions according to the disclosure may impart a film or coating on a substrate, such as on the hair or on the hand during or after application to the hair. A film formed by the composition may, surprisingly, be clean-feeling and not sticky, as with traditional hair styling compositions. Also surprisingly, the composition may impart a film on the hair that leaves the hair relatively natural and clean-feeling, yet has a flexible coating, leaving little to no residue, allows the hair to be bouncy and springy with little to no frizz or flaking, may impart relatively high definition with individualized curls, style control, volume, and shine, and/or may allow for relatively long-lasting hold and style memory. Furthermore, in at least certain embodiments according to the disclosure, the compositions are not sticky or tacky. A user of hair compositions according to various embodiments described herein may thus feel that the composition is not perceptible or is “invisible,” yet still effectively style and/or hold the hair. Additionally, the compositions may have effective hair styling and/or hold properties, even in conditions of high, or relatively high, humidity. In at least certain embodiments according to the disclosure, the compositions may be quick-drying, which may allow drying and/or styling time to be reduced, as well as further improve ease of styling and curl retention.

Furthermore, as described, compositions prepared according to various embodiments may provide for varying degrees of hold to be imparted to a hair style. By way of non-limiting example only, in order to obtain a spiky look to hair of a very short length, a high level of styling hold (“strong hold”) may be desirable. Or, as a further non-limiting example, in order to obtain a flowing look or to maintain hair curls for hair of medium length or longer length, a light to medium level of style hold may be desirable. By altering the weight ratio of the latex polymer and the polycarbodiimide, it is possible to formulate compositions having high levels of style hold, medium to high levels of style hold, medium levels of style hold, or light to medium levels of style hold.

In certain embodiments, when the weight ratios polycarbodiimide to latex polymer is from about 1:50 about 10:1 or about 1:40 to about 10:1 or about 20:1 to about 10:1 or about 1:10 to about 10:1 or about 5:1 to about 1:5 or about 2:1 to about 1:2 or at about 1:1, various degrees of styling hold may be obtained ranging from light to strong hold.

In some particular embodiments, the compositions include weight ratios of latex polymer to polycarbodiimide about 1:1 to about 10:1, and in some embodiments from about 2:1 or about 3:1 or about 4:1 or about 5:1. In some embodiments, at least two different latex polymers are present in the compositions, wherein the weight ratios of each of the latex polymers to the polycarbodiimide are from about 1:1:1 to about 5:1:1 and in some embodiments from about 2:1:1 or from about 3:1:1 or from about 4:1:1.

In at least certain embodiments, a film formed by the compositions described herein may be clear and/or stable. In addition, hair styled or treated with compositions according to the disclosure may, in at least certain exemplary embodiments, be hydrophobic, and/or may appear less frizzy and/or may be less prone to breakage, relative to hair subjected to the same conditions but not having been styled or treated with a composition according to the disclosure.

It should be noted, however, that compositions and films, as well as hair to which the composition or film has been applied, according to the disclosure may not have one or more of the herein-referenced properties, yet are intended to be within the scope of the disclosure.

Also disclosed herein are methods for styling the hair, said methods comprising applying a composition according to the disclosure to the hair, either before, during, or after styling the hair. one or more steps of treating the hair with an external stimuli, such as heat, before, during, or after the composition has been applied to the hair are also contemplated.

Instructions for applying the composition of the present invention onto keratinous substrates such as hair on the head or eyelashes may comprise directions of use of the composition for the end-user to follow. The end-user may be a consumer or cosmetologist or salon hair dresser. Directions may comprise instructing the end-user to take an amount of the composition in sufficient quantity such that the composition adequately covers the hair fibers and imparts the desired shape or style or hold to the hair fibers. Directions may additionally instruct the end-user to use a device such as a comb, brush (e.g., hair brush or brush wand), flat iron plates, blow dryer or the fingers for shaping or styling the hair or for separating the fibers of the hair. Directions may also additionally instruct the end-user to apply heat to the hair such as by blow drying the hair or using a heating device on the hair.

Instructions for applying the composition of the present invention onto keratin fibers such as hair may appear on the container (such as can, bottle or jar) holding the composition of the present invention or on the box or carton or other packaging comprising the container holding said composition.

Another embodiment of the present invention is method protecting a keratinous fiber chosen from hair comprising applying to the keratinous fiber the composition of the present invention in an amount effective to protect or repair said keratinous fiber before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

The compositions described above are useful for application onto keratinous substrates such as hair on the head of human individuals.

Thus, the compositions of the present invention can be made into various cosmetic products such hair care products, hair styling products and make up products.

Representative types of hair care compositions, including hair cosmetic and styling compositions, of the present invention include compositions for shaping the hair, maintaining the shape of the hair, styling products (e.g., gels, creams, milks, pastes, waxes, ointments, serums, foams, hair lotions, mousses, pump-sprays, non-aerosol sprays and aerosol sprays), conditioning or protection from heat damage, leave-in hair treatments, rinse-off hair treatments, combination shampoo/styling compositions and hair volumizing compositions.

The compositions of the present invention can be in the form of an aqueous composition or an emulsion, such as a lotion or cream.

In one embodiment, the composition of the present invention is in the form of a non-aerosol spray, in certain embodiments containing a volatile organic solvent/compound.

In one embodiment, the composition of the present invention is in the form of a cream. In another embodiment, the composition of the present invention is in the form of an aqueous composition.

The compositions may be packaged in various forms, especially in a tube, a jar or bottles, in pump bottles, in squeeze bottles, or in aerosol containers so as to apply the composition in vaporized form or in the form of a mousse. The compositions may also impregnate applicators, especially gloves or wipes.

The composition may be applied by hand, with an applicator nozzle or actuator pump, with a container equipped with a pump, an applicator and a dispensing comb, or with an insoluble substrate impregnated with the composition.

As used herein, the process and composition disclosed herein may be used on the hair that has not been artificially dyed, pigmented or permed.

As used herein, the process and composition disclosed herein may be also used on the hair that has been artificially dyed, pigmented or permed.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

Procedure for Preparation of Hair Composition (for Hair Repair or Hair Styling)

Stock solutions of each phase were generated by stirring a selected amount or percentage by weight of active RM (raw material) in the selected solvent (either water, or mixture of water and organic solvent such as Isododecane (IDD) or ethanol, or organic solvent). Just before application, the desired ratio of parts (typically 1:1 actives) were weighed into a vial and mixed to create the hair treatment solution. When the two phases comprise an aqueous phase and a non-aqueous phase, the two phases in the vial were agitated or shaken to create an emulsion. Typically, 0.5 to 1 g of product was applied to 0.5 to 1 g of hair. In the case of 2-step treatments, stock solutions are applied directly to hair without pre mixing.

### EXAMPLES

Raw Materials Employed in the Examples I and II

| Designations | Ingredient Information |
|---|---|
| Latex 1 | Acrylates Copolymer (INCI name), Commercially |
| | available as LUVIFLEX SOFT from the supplier |
| | BASF |
| Latex 2 | Acrylates Copolymer (INCI name); Commercially |
| | available as DAITOSOL 3000SLPN from the supplier |
| | Daito Kasei Kogyo |
| Latex 3 | Acrylates/Ethylhexyl Acrylate Copolymer (INCI |
| | name); Commercially available as DAITOSOL |
| | 5500GM from the supplier Daito Kasei Kogyo |
| Latex 4 | Polyacrylate-32 (INCI name); Commercially available |
| | as FIXATE DESIGN POLYMER from the supplier |
| | Lubrizol |
| Latex 5 | POLYQUATERNIUM-91 (and) POLYACRYLATE- |
| | 15, Commercially available as Syntran PC 5330 |
| Latex 6 | Polyacrylate-15, Commercially available as Syntran |
| | PC 5500 |
| Latex 7 | 24.3% active weight, Polyacrylate 21 (and) |
| | Acrylates/Dimethylaminoethyl Methacrylate |
| | Copolymer; Commercially available as Syntran PC |
| | 5100 |
| Latex 8 | acrylate grafted olefin polymer - |
| | ACRYLATES/ETHYLHEXYL ACRYLATE/HEMA |
| | COPOLYMER (and) |
| | ACRYLATES/DIETHYLAMINOETHYL |
| | METHACRYLATE/ETHYLHEXYL ACRYLATE |
| | COPOLYMER (and) ISODECETH-6 (and) |
| | CAPRYLYL GLYCOL (and) SODIUM LAURETH |
| | SULFATE; Commercially available as Syntran PC |
| | 5775 |
| Polycarbodiimide | Commercially available as CARBODILITE SV-02 |
| SV02 (“pCDI | from the supplier Nisshinbo |
| SV02”) | |

### Examples I

### A. Test Procedures

1) Determination of the Physical Properties of Films from Inventive Combination

Film Plating

The latex film is obtained by allowing a 30 g water solution containing 4 g of the latex+polycarbodiimide polymer (active solid amount) at various ratios to dry slowly in a 100 ml PFA Petri dish (100 mm diameter×15 mm height) at room temperature for at least 3 days. The latex film formers are the ones have high Tg (>60° C.), medium Tg (10-60° C.) and low Tg (<10° C.).

Film Measurement

The latex film, with known dimensions (length, width, thickness), is mounted on the Q800 Dynamic Mechanical Analysis from TA Instrument, and tested in a DMA Control Force mode. The stress/strain test is obtained using the following procedure:

a. Initial Strain: 0.01%

b. Isothermal: 25° C.

c. Strain Rate: 5%/min

d. Final Strain: 50%

From the stress/strain curve, the Young's Modulus is calculated as the slope of the linear portion at about 0.01% Strain to about 0.1% Strain, and the strain at indicated stress. A high Young's Modulus represents a hard film, a lower Young's Modulus represents a softer film. An increase of strain indicated a more flexible film.

2) Determination of the Mechanical Property of Hair Treated with Inventive Combination

Hair Treatment

A strip of normal hair (from IHIP, 1 cm in width, 15 cm long, about 2-2.5 g of hair) was treated with the inventive solution (0.5 g of solution/g hair). The hair was combed through until the solution was uniformly distributed over the surface of the tress. The treated hair, in a straight configuration, was allowed to dry overnight at room temperature.

Hair Measurement

Three-point bending measurements are conducted using a texture analyzer (Model TA-XTPlus, Texture Technologies Corporation) equipped with a hair mounting accessory as described in J. Cosmet. Sci., 53, 345-362 (November/December 2002). The cantilever bending experiment consists of the following sequence of steps: the hair tress is placed on a 2-point of 6 cm width, and the probe, representing the third point, comes down at the middle of the hair tress and performs ten 10-mm deformations of the hair tress. The testing protocol is:

Test mode=Compression

Pre-test speed=2 mm/sec

Test speed=2 mm/sec

Post-test speed=2 mm/sec

Target mode=Distance

Distance=10 mm

Count=10

Trigger type=Auto (Force)

Trigger force=1 g

After finishing 10 cycles of bending, a plot of force as a function of distance of 10 deformations is generated. From the plot, the maximum force in the first deformation (FMax) was determined.

A high maximum force represents the hair being stiff with strong hold; a lower maximum force represents the hair was softer with weaker hold.

The results are obtained from the average of triplicate experiments.

3) Determination of the High Humidity Curl Retention

Hair Treatment

Regular bleached hair swatch (from IHIP, 14.5 cm long, about 0.5 g) was treated with a solution of the inventive combination (0.5 g solution/g hair). The hair was combed until the solution is uniformly distributed over the hair swatch surface. The treated hair was then rolled onto a spiral rod (0.5 in diameter) and (a) allowed to dry at room temperature overnight; or b) cure in a 60° C. oven for 30 min then equilibrate at room temperature overnight.

Curl Retention Measurement

The coiled hair was removed from the rod and placed in the humidity chamber at 90% RH, 25° C. for 24 hours. The Curl Retention was calculated as: (Lo−Lf)/(Lo−Li)×100, wherein Lo=Fully extended hair length (14.5 cm), Li=Initial coiled hair length before humidity exposure, and Lf=Final hair length after 24 hr exposure.

4) Determination of the Hydrophobicity

Hair Treatment

Swatches of twice-bleached hair (from IHIP, 0.5 cm wide, 0.75 g) were treated with various solutions of latices and polycarbodiimides, then placed in a 50° C. oven for 30 minutes, then dried overnight at room temperature. The following morning, they were washed with a conventional sulfate-based shampoo (DOP shampoo), dried in a helmet dryer then measured for residual hydrophobicity (i.e. hydrophobicity that survived the shampoo/wash cycle).

Hydrophobicity Measurement

Hydrophobicity of each treated swatch was measured via contact angle measurements using Biolin Scientific Contact Angle Tensiometer, Model C204A. A bundle of 30-50 fibers was clamped to create a flat surface. A 3-5 uL drop of DI H₂O was placed on the fiber surface and the contact angle was measured for 10 seconds. The values reported below are an average of 3 measurements using the contact angle at 10 seconds.

### B. Examples I. Physical Properties

1) High Tg Latex+Polycarbodiimide

Clear films were obtained from the combination of Latex 1 (Tg˜128° C., 27% active) and pCDI 1 (40% active) at various ratios. The composition and physical properties of the films are shown below.

TABLE 1

| | | | Young's | % |
|---|---|---|---|---|
| Ingredients | Latex 1 | pCDI SV02 | Modulus | strain@0.6 |
| and ratios | (g AM*) | (g AM*) | (Mpa) | Mpa Stress |
| Latex 1 | 4 | 0 | 2500 | 0.007 |
| 10:1 | 3.63 | 0.37 | 4834 | 0.002 |
| 5:1 | 3.33 | 0.67 | 3053 | 0.01 |
| 1:1 | 2 | 2 | 1738 | 0.04 |
| 1:5 | 0.67 | 3.33 | 850.4 | 0.08 |
| 1:10 | 0.37 | 3.63 | 562.7 | 0.1 |
| Polycarbodiimide | 0 | 4 | Does not | |
| SV02 | | | form a film | |

*AM = active material

The results show that at lower amounts of the polycarbodiimide, the film was harder and less flexible; as the amount of polycarbodiimide increased with respect to the amount of the latex polymer, the film became softer and more flexible. These results indicate that for high Tg latex polymers which generally form harder and less flexible films by themselves, the addition of polycarbodiimide affects or “tunes” the physical properties of the latex polymer such that at varying amounts of the polycarbodiimide, a range of physical properties with respect to flexibility and softness/hardness of a film can be obtained. This translates to the ability of the inventive compositions to form films or coatings on hair that can provide different types of styling or shaping hold to the hair (medium to strong hold).

2) Medium Tg Latex+Polycarbodiimide

Clear films are obtained from the combination of Latex 2 (Tg˜35° C., 30% active) and pCDI SV02 (40% active) at various ratios. The composition and physical properties of the film are shown below.

TABLE 2

| | | pCDI 1 | Young's | % |
|---|---|---|---|---|
| Ingredients | Latex 2 (g | (g | Modulus | strain@0.01 Mpa |
| and ratios | AM) | AM) | (Mpa) | Stress |
| Latex 1 | 4 | 0 | 24.5 | 0.086 |
| 10:1 | 3.63 | 0.37 | 25.4 | 0.24 |
| 5:1 | 3.33 | 0.67 | 26.5 | 0.33 |
| 1:1 | 2 | 2 | 48.5 | 0.12 |
| 1:5 | 0.67 | 3.33 | 1 | □50 |
| 1:10 | 0.37 | 3.63 | 0.05 | □50 |
| Polycarbodiimide | 0 | 4 | Does not | — |
| SV02 | | | form a film | |

The results show that at lower amounts of the polycarbodiimide, the film was harder and less flexible; as the amount of polycarbodiimide increased with respect to the amount of the latex polymer, the film became softer and more flexible. These results indicate that for medium Tg latex polymers, the addition of polycarbodiimide affects or “tunes” the physical properties of the latex polymer such that at varying amounts of the polycarbodiimide, a range of physical properties with respect to flexibility and softness/hardness of a film can be obtained. This translates to the ability of the inventive compositions to form films or coatings on hair that can provide different types of styling or shaping hold to the hair ranging from light to medium hold).

3) Low Tg Latex+Polycarbodiimde

Clear films are obtained from the combination of Latex 3 (Tg˜67° C., 55% active) and polycarbodiimide 1 (40% active) at various ratios. The composition and physical properties of the film are shown below.

TABLE 3

| | | pCDI | | % |
|---|---|---|---|---|
| | Latex 3 | SV02 | | strain@0.01 Mpa |
| | (g AM) | (g AM) | Young's Modulus (Mpa) | Stress |
| Latex 3 | 4 | 0 | Not measurable due | |
| | | | to viscous film | |
| 10:1 | 3.63 | 0.37 | 0.58 | 2.75 |
| 5:1 | 3.33 | 0.67 | 5.6 | 3.67 |
| 1:1 | 2 | 2 | 0.33 | >50 |
| 1:5 | 0.67 | 3.33 | Does not form a film | — |
| 1:10 | 0.37 | 3.63 | Does not form a film | — |
| pCDI | 0 | 4 | Does not form a film | — |
| SV02 | | | | |

The results show for a low Tg latex polymer which forms a viscous film at room temperature, the addition of different amounts of polycarbodiimide (up to a ratio of 1:1) helped to obtain a good film with various physical properties such as different degrees of flexibility and softness of the film. This translates to the ability of the inventive compositions to form films or coatings on hair that can provide different degrees of light styling or shaping hold to the hair.

II. Mechanical Property of Hair

Normal virgin hair swatches (about 2 g) were treated with aqueous solutions containing 3% Latex 2 and 0 to 6% pCDI SV02 (2 g product/g hair). The treated hair swatches were dried at room temperature overnight. A 3-point bending experiment was performed on these hair swatches to determine the maximum force (FMax) to bend the hair 10 cm downward.

TABLE 4
Treatments

| | % | % | |
|---|---|---|---|
| Treatment | Latex | Polycarbodiimide | Ratio |
| Hair treated with latex alone | 3 | 0 | 0 |
| Hair treated with latex/polycarbodiimide | 3 | 0.06 | 50:1 |
| Hair treated with latex/polycarbodiimide | 3 | 0.15 | 20:1 |
| Hair treated with latex/polycarbodiimide | 3 | 0.3 | 10:1 |
| Hair treated with latex/polycarbodiimide | 3 | 0.6 | 5:1 |
| Hair treated with latex/polycarbodiimide | 3 | 1.5 | 2:1 |
| Hair treated with latex/polycarbodiimide | 3 | 3 | 1:1 |
| Hair treated with latex/polycarbodiimide | 3 | 6 | 1:2 |

TABLE 5
3-Point Bending results

| Treatment | FMax (g) |
|---|---|
| 3% Latex | 119 |
| 0.06% Polycarbodiimide | 8 |
| 50:1 Latex:Polycarbodiimide | 119 |
| 0.15% Polycarbodiimide | 12 |
| 20:1 Latex:Polycarbodiimide | 129 |
| 0.3% Polycarbodiimide | 18 |
| 10:1 Latex:Polycarbodiimide | 140 |
| 0.6% Polycarbodiimide | 41 |
| 5:1 Latex:Polycarbodiimide | 189 |
| 1.5% Polycarbodiimide | 117 |
| 2:1 Latex:Polycarbodiimide | 296 |
| 3% Polycarbodiimide | 114 |
| 1:1 Latex:Polycarbodiimide | 467 |
| 6% Polycarbodiimide | 170 |
| 1:2 Latex:Polycarbodiimide | 795 |

The results above show that the polycarbodiimide helps to increase the hold of the latex polymer on hair and that the hold (based on stiffness of the hair) increases with increasing amounts of the polycarbodiimide.

III. High Humidity Curl Retention of Hair (HHCR study)

1) Medium Tg Latex+Polycarbodiimde

Hair swatches were treated with solutions of Latex 2 and Polycarbodiimide SV02 at various ratios, then dried at room temperature overnight. Their high humidity curl retention results are shown below.

TABLE 6

| | Latex 2 | pCDI SV02 | |
|---|---|---|---|
| | (g AM) | (g AM) | HHCR (%) |
| Latex 2 | 2 | 0 | 48 |
| 1:1 | 1 | 1 | 83 |
| 1:5 | 0.33 | 1.67 | 83 |
| 1:10 | 0.18 | 1.82 | 67 |
| pCDI SV02 | 0 | 2 | 62 |

2) Low Tg Latex+Polycarbodiimde

Hair swatches were treated with 2% solutions of Latex 3 and Polycarbodiimide SV02 at various ratios, then cured at 60° C. oven for 30 min, followed by further drying at room temperature overnight. Their high humidity curl retention results are shown below.

TABLE 7

| | Latex 3 (g AM) | pCDI SV02 (g AM) | HHCR (%) |
|---|---|---|---|
| Latex 3 | 2 | 0 | 50 |
| 10:1 | 1.82 | 0.18 | 74 |
| 5:1 | 1.67 | 0.33 | 79 |
| pCDI SV02 | 0 | 2 | 58 |

The results in Tables 6 and 7 show higher HHCR values for the latex polymer/polycarbodiimide compositions as compared to the HHCR values for each of the latex polymer or polycarbodiimide alone. This indicates that the inventive compositions impart high humidity resistance property to the hair such that hair is able to maintain significantly better styling or shaping hold to the hair even in high humidity conditions.

IV. Hydrophobicity of Hair

1) Medium Tg Latex+Polycarbodiimde

Hair swatches are treated as described above with 2% solutions of Latex 4 and Polypolycarbodiimide SV02 or mixtures of the two. Hydrophobicity measurements were then taken.

TABLE 8

| Treatment | Contact Angle (°) |
|---|---|
| 2% Latex | 0 |
| 2% Latex + 2% pCDI SV02 | 105 |
| 2% pCDI SV02 | 0 |

2) Low Tg Latex+Polycarbodiimde

Hair swatches are treated as described above with 2% solutions of Latex 3 and Polypolycarbodiimide SV02 or mixtures of the two. Hydrophobicity measurements were then taken.

TABLE 9

| Treatment | Contact Angle (°) |
|---|---|
| 2% Latex | 0 |
| 2% Latex + 2% pCDI SV02 | 55 |
| 2% pCDI SV02 | 0 |

The contact angle measurements above show that the combination of polycarbodiimide with the latex polymer resulted in increased hydrophobicity imparted to the hair. In contrast, the polycarbodiimide and the latex polymer did not impart hydrophobicity to hair as shown by the zero contact angles.

In summary, the above examples show that the combination of the latex polymer and the polycarbodiimide yield films with unique properties (physical, mechanical, hydrophobicity). At the same time, the results from the examples above indicate that the interaction between the latex polymer, polycarbodiimide and the hair imparts benefits to the hair such as styling or shaping performance that can be controlled to yield light to medium to strong hold, increased hydrophobicity and durable shape or style such as curl against high humidity conditions, and bending force properties that help keep the shape or configuration of hair, These results offer manufacturers and users of cosmetic products the ability and the tools to control and to improve, as desired, the stiffness or hardness/softness, the rigidity/flexibility, the shape control/style memory, the resistance/resiliency against external factors such as low/high humidity conditions, chemical treatments, shampoo/wash treatments, and deformation produced by grooming, bodily motion, wind, head covers, on the hair shape/style of individuals with diverse types of hair (e.g., short to long hair, straight to curly hair).

### Examples II

The cosmetic application of polycarbodiimide and latex containing carboxylic acid association for hair applications

A. Testing Procedures

Procedure for Durability Determination Using High Humidity Curl Retention (HHCR) Test

Hair Treatment

Regular bleached hair swatch (from IHIP, 13.5 cm long, about 0.5 g weight) is treated with solutions of 4% by weight of active material of test solutions (0.5 g solution/g hair). The hair is combed until the solution is uniformly distributed over the hair swatch surface. The treated hair is then rolled onto a spiral rod (0.5 in diameter) and allowed to:

a) Dry at room temperature overnight or

b) Dry in a 50 C oven for 30 minutes and then dried at room temperature overnight.

Curl Retention Measurement

The coiled hair is removed from the rod and placed in the humidity chamber at 90% RH, 40° C. for 5 hours. % Curl Retention was calculated using the formula below:

% Curl Retention=(Lo−Lt)*100(Lo−Li)

Where: Lo=Original hair length (fully extended hair length)
Li=Initial hair length (length of hair before humidity exposure)
Lt=Length of hair after 5 hr humidity exposure

1a) High Humidity Curl Retention of Hair Treated with a cationic latex polymer having a carboxylic acid group, Syntran PC 5330 (POLYQUATERNIUM-91 (and) POLYACRYLATE-15) and Polycarbodiimide V02-L2 Five hair swatches were treated with the solutions below (0.5 g/g of product):

The swatches were then dried overnight around the curling rods. The high humidity curl retention results are shown below.

TABLE 10

| | % curl |
|---|---|
| Treatment* | after 5 hrs |
| 4% by weight Syntran 5330 | 16% |
| 4% by weight 1:1 (Syntran 5330 + Polycarbodiimide V02-L2) | 59% |
| 4% by weight 3:1 (Syntran 5330 + Polycarbodiimide V02-L2) | 62% |
| 4% by weight 5:1 (Syntran 5330 + Polycarbodiimide V02-L2) | 80% |
| 4% by weight Polycarbodiimide V02-L2 | 17% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

1b) High Humidity Curl Retention of Hair Treated with a cationic latex polymer having a carboxylic acid group, Syntran PC 5330 and Polycarbodiimide SV02 Five hair swatches were treated with the solutions below (0.5 g/g of product):

The swatches were then dried overnight around the curling rods. The high humidity curl retention results are shown below.

TABLE 11

| | % curl after |
|---|---|
| Treatment* | 5 hrs |
| 4% by weight Syntran 5330 | 16% |
| 4% by weight 1:5 (Syntran 5330 + Polycarbodiimide SV02) | 22% |
| 4% by weight 1:1 (Syntran 5330 + Polycarbodiimide | 43% |
| 4% by weight 5:1 (Syntran 5330 + Polycarbodiimide SV02) | 60% |
| 4% by weight Polycarbodiimide SV02 | 12% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

2a) High Humidity Curl Retention of Hair Treated with a cationic latex polymer having a carboxylic acid group Syntran PC 5500 (Polyacrylate-15) and Polycarbodiimide V02-L2

Five hair swatches were treated with the solutions below (0.5 g/g of product):

The swatches were then dried overnight around the curling rods. The high humidity curl retention results are shown below.

TABLE 12

| | % curl |
|---|---|
| Treatment* | after 5 hrs |
| 4% by weight Syntran 5500 | 34% |
| 4% by weight 1:1 (Syntran 5500 + Polycarbodiimide V02-L2) | 53% |
| 4% by weight 3:1 (Syntran 5500 + Polycarbodiimide V02-L2) | 62% |
| 4% by weight 5:1 (Syntran 5500 + Polycarbodiimide V02-L2) | 62% |
| 4% by weight Polycarbodiimide V02-L2 | 17% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

2b) High Humidity Curl Retention of Hair Treated a cationic latex polymer having a carboxylic acid group Syntran PC 5500 (Polyacrylate-15) and Polycarbodiimide SV02

Five hair swatches were treated with the solutions below (0.5 g/g of product):

The swatches were then dried overnight around the curling rods. The high humidity curl retention results are shown below.

TABLE 13

| | % curl after |
|---|---|
| Treatment* | 5 hrs |
| 4% by weight Syntran 5500 | 34% |
| 4% by weight 1:5 (Syntran 5500 + Polycarbodiimide SV02) | 40% |
| 4% by weight 1:1 (Syntran 5500 + Polycarbodiimide SV02) | 49% |
| 4% by weight 5:1 (Syntran 5500 + Polycarbodiimide SV02) | 80% |
| 4% by weight Polycarbodiimide SV02 | 32% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

3) High Humidity Curl Retention of Hair Treated with an amphoteric latex polymer having a carboxylic acid group, Syntran PC 5100 (24.3% active weight, Polyacrylate 21 (and) Acrylates/Dimethylaminoethyl Methacrylate Copolymer) and Polycarbodiimide V02-L2

Five hair swatches were treated with the solutions below (0.5 g/g of product):

The swatches were then dried overnight around the curling rods. The high humidity curl retention results are shown below.

TABLE 14

| | % curl |
|---|---|
| Treatment* | after 5 hrs |
| 4% by weight Syntran 5100 | 70% |
| 4% by weight 1:1 (Syntran 5100 + Polycarbodiimide V02-L2) | 88% |
| 4% by weight 3:1 (Syntran 5100 + Polycarbodiimide V02-L2) | 84% |
| 4% by weight 5:1 (Syntran 5100 + Polycarbodiimide V02-L2) | 82% |
| 4% by weight Polycarbodiimide V02-L2 | 17% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

4) High Humidity Curl Retention of Hair Treated with an amphoteric latex polymer having a carboxylic acid group, Syntran PC 5775 (acrylate grafted olefin polymer—ACRYLATES/ETHYLHEXYL ACRYLATE/HEMA COPOLYMER (and) ACRYLATES/DIETHYLAMINOETHYL METHACRYLATE/ETHYLHEXYL ACRYLATE COPOLYMER (and) ISODECETH-6 (and) CAPRYLYL GLYCOL (and) SODIUM LAURETH SULFATE and Polycarbodiimide V02-L2

Five hair swatches were treated with the solutions below (0.5 g/g of product):

The swatches were then dried overnight around the curling rods. The high humidity curl retention results are shown below.

TABLE 15

| | % curl |
|---|---|
| Treatment* | after 5 hrs |
| 4% by weight Syntran 5775 | 68% |
| 4% by weight 1:1 (Syntran 5775 + Polycarbodiimide V02-L2) | 89% |
| 4% by weight 3:1 (Syntran 5775 + Polycarbodiimide V02-L2) | 88% |
| 4% by weight 5:1 (Syntran 5775 + Polycarbodiimide V02-L2) | 82% |
| 4% by weight Polycarbodiimide V02-L2 | 17% |

*amounts of the ingredients in the treatment solutions are expressed in percent total weight of active material in water and ratios are weight ratios

The results indicate that hair treated with the inventive compositions at room temperature has increased percent curl retained indicating higher resistance to high humidity and high temperature compared to hair treated with either material alone. The inventive composition displayed a higher styling property over time and at high humidity condition as indicated by the higher curl retention value. In contrast, the hair treated with either material alone demonstrated a lower curl retention value, indicating less styling hold over time and at high humidity.

5) Adhesion+Conditioning Testing of Latex with Polycarbodiimide V02-L2

4% solutions were poured into polypropylene weighboats (similar surface energy to that of hair). When the resulting films were peeled, the adhesion value was assessed based on the difficulty of peeling the films (with 1 being the easiest and 5 being the hardest to peel). Conditioning is defined as the ease of passing a brush through the treated hair. Hair is treated with 0.25 g of product/g of hair.

TABLE 16

| | Adhesion | Conditioning |
|---|---|---|
| Latex | (in weigh boat) | (Ease of Brushing) |
| 4% Syntran 5330 | 1 | 5 |
| 4% @ 1:1Syntran 5330 + V02-L2 | 4 | 5 |
| 4% Syntran 5500 | 1 | 5 |
| 4% @1:1 Syntran 5500 + V02-L2 | 4 | 4 |
| 4% Syntran 5100 | 2 | 3 |
| 4% @ 1:1 Syntran 5100 + V02-L2 | 4 | 3 |
| 4% V02-L2 | 4 | 1 |

*amounts of the ingredients in the treatment solutions are expressed in percent total weight of active material in water and ratios are weight ratios

The adhesion properties for latex polymers alone were very poor. In contrast, the adhesion properties increased with the combination of latex polymer and polycarbodiimide V02-L2 (inventive compositions). In addition, while the conditioning and ease of brushing with polycarbodiimide alone was very difficult, the addition of a latex polymer to the polycarbodiimide resulted in comparable conditioning as that obtained from the latex polymer alone (indicates that there is no significant negative impact on the conditioning properties imparted by the latex polymers) Thus, the inventive compositions containing the association of latex+polycarbodiimide provided both conditioning and adhesion benefits (best balance of conditioning and adhesion).

5) Dynamic Mechanical Analysis (DMA) Results

Determination of the physical properties of films formed by the inventive combinations

Film Plating

The film was obtained by allowing a 30 g water solution containing the latex and polycarbodiimide polymers (4 g total active solid amount at various ratios to dry slowly in a 100 ml PFA Petri dish (100 mm diameter×15 mm height) at room temperature for at least 3 days. The latex film formers are the ones having high Tg (>60° C.), medium Tg (10-60° C.) and low Tg (<10° C.).

Film Measurement

The film, with known dimensions (length, width, thickness), was mounted on the Q800 Dynamic Mechanical Analysis from TA Instrument, and tested in a DMA Temperature Ramp mode. The stress/strain test was obtained using the following procedure:

Temperature range: 0-150° C.

Temp Ramp Rate: 3° C./min

From the curve, the Storage Modulus at 25° C. was determined. A high storage Modulus represents a hard film, a lower Storage Modulus represents a softer film.

TABLE 17

| | Young's Modulus |
|---|---|
| Syntran PC5500 | 603 Mpa |
| Syntran PC5500:polycarbodiimide SV02, weight | 673.5 Mpa |
| ratio of 1:1 | |
| Syntran 5100 | 243 Mpa |
| Syntran 5100:polycarbodiimide V02-L2, weight | 401 Mpa |
| ratio of 5:1 | |
| Syntran 5775 | 86.6 Mpa |
| Syntran 5775:polycarbodiimide V02-L2, weight | 180.5 Mpa |
| ratio of 5:1 | |
| polycarbodiimide V02-L2 | Not a film former |

The results indicate that films of the inventive compositions have increased in stiffness, compared to either material alone. Incorporating polycarbodiimide into the latex polymer solution produced a coating with increase stiffness through crosslinking of the polymers. This increased film stiffness translates to improved shaping performance of the coating on hair.

6a) Frizz Control Blowdry—Example with latex associations (at least 2 latexes).

The two latex polymers were tested: Syntran PC 5330 is cationic latex polymer, Daitosol 5000 AD is Nonionic Latex.

Hair swatches (IHIP Frizzy hair) were washed with DOP shampoo containing mainly sodium laureth sulfate, coco-betaine and water, then the products were applied on the hair (0.1 g of product/g of hair). The swatches were blow dried (rough dry for 30 seconds, to remove excess moisture for 30 seconds with blowdrier, then with round brush for 1 minute to style). Hair was assessed by ranking the ease of brushing during the blowdrying steps.

Hair swatches were then mounted on a shaking apparatus inside the humidity chamber (to mimic real life movement) for 5 hours at high humidity and temperature. The frizz control was then assessed by expert evaluators by ranking.

TABLE 18
Ratings. 1-low performance and 5-high performance.

| | Syntran | Daitosol | | Ease of | |
|---|---|---|---|---|---|
| | PC5330 | 5000 AD | V02-L2 | Brushing | Frizz Control |
| | (% | (% | (% | during | after 5 hours |
| | active) | active) | active) | blowdry | 90% RH 40 C. |
| 3% Syntran PC 5330 | 3 | 0 | 0 | 5 | 2 |
| 3% Daitosol 5000 AD | 0 | 3 | 0 | 1 | 1 |
| 3% 1:1 Daitosol 5000 | 0 | 1.5 | 1.5 | 1 | 1 |
| AD + V02L2 | | | | | |
| 3% 3:1 Syntran PC | 2.25 | 0.75 | 0 | 4 | 3 |
| 5330 + Daitosol 5000 | | | | | |
| AD | | | | | |
| 3% 1:1 Syntran | 1.5 | 0 | 1.5 | 4 | 3 |
| PC5330 + V02L2 | | | | | |
| 3% 3:1:1 Syntran PC | 1.5 | 0.5 | 0.5 | 4 | 5 |
| 5330 + Daitosol 5000 | | | | | |
| AD + V02L2 | | | | | |
| 3% V02L2 | 0 | 0 | 3 | 1 | 1 |

*amounts of the ingredients in the treatment solutions are expressed in percent total weight of active material in water and ratios are weight ratios

The results indicate that hair treated with the composition containing the combination of the cationic and non-ionic latex polymers and Polycarbodiimide (2 latex-pCDI combination) had exhibited increased ease of brushing, initial frizz control after brushing and lasting frizz control effects when exposed to high humidity conditions as compared to hair treated with either material alone or the combination of the nonionic latex polymer and Polycarbodiimide. The 2 latex-pCDI combination also performed comparably to the 2 latex combination with respect to ease of brushing and initial frizz control and provided increased lastingness of the frizz control performance in high humidity conditions.

6b) Frizz Control Airdry—Example with latex associations (at least 2 latexes).

The two latex polymers were tested: Syntran PC 5330 is cationic latex polymer, Daitosol 5000 AD is Nonionic Latex.

Hair swatches (IHIP Frizzy hair) were washed with DOP shampoo containing mainly sodium laureth sulfate, coco-betaine and water, then the products were applied on the hair (0.5 g of product/g of hair). Hair was assessed by ranking during application of product for ease of distributing solution evenly through the swatch. The hair swatches were combed twice. Hair was allowed to dry overnight at room temperature.

Hair swatches were then mounted on a shaking apparatus inside the humidity chamber (to mimic real life movement) for 5 hours at high humidity and temperature. The frizz control was then assessed by expert evaluators by ranking.

TABLE 19
Ratings. 1-low performance and 5-high performance.

| | Syntran | Daitosol | | | Frizz Control |
|---|---|---|---|---|---|
| | PC5330 | 5000 AD | V02-L2 | | after 5 hours |
| | (% | (% | (% | Ease of | 90% RH |
| | active) | active) | active) | distribution | 40 C. |
| 3% Syntran PC 5330 | 3 | 0 | 0 | 5 | 1 |
| 3% Daitosol 5000 AD | 0 | 3 | 0 | 1 | 2 |
| 3% 1:1 Daitosol 5000 | 0 | 1.5 | 1.5 | 1 | 3 |
| AD + V02L2 | | | | | |
| 3% 3:1 Syntran PC | 2.25 | 0.75 | 0 | 4 | 3 |
| 5330 + Daitosol 5000 | | | | | |
| AD | | | | | |
| 3% 1:1 Syntran | 1.5 | 0 | 1.5 | 4 | 3 |
| PC5330 + V02L2 | | | | | |
| 3% 3:1:1 Syntran PC | 1.5 | 0.5 | 0.5 | 4 | 5 |
| 5330 + Daitosol 5000 | | | | | |
| AD + V02L2 | | | | | |
| 3% V02L2 | 0 | 0 | 3 | 1 | 1 |

*amounts of the ingredients in the treatment solutions are expressed in percent total weight of active material in water and ratios are weight ratios

The results indicate that following the air dry method, hair treated with the composition containing the combination of the cationic and non-ionic latex polymers and Polycarbodiimide (2 latex-pCDI combination) had high ratings for all three attributes of increased ease of brushing, initial frizz control after brushing and lasting frizz control effects when exposed to high humidity conditions.

7) Wash Study—Frizz Control in high humidity after shampoo

Swatches treated with the various treatments (0.25 g of solution/g hair) were allowed to airdry overnight. Then washed once with DOP shampoo. Hair was dried overnight at RT and then placed in the humidity chamber at 80% humidity 25 C for 8 hours

TABLE 20
Ratings by expert evaluator. 1-Low 5-High

| | | | | Humidity |
|---|---|---|---|---|
| | | | | Resistance |
| | | | | after Shampoo |
| | | | | as measured |
| | | Daitosol | | by ranking |
| | Syntran | 5000 | | of antifrizz |
| | PC5330 | AD | V02-L2 | properties |
| 3% Syntran PC 5330 | 3 | 0 | 0 | 1 |
| 3% Daitosol 5000 AD | 0 | 3 | 0 | 1 |
| 3% 1:1 Daitosol 5000 AD + | 0 | 1.5 | 1.5 | 2 |
| V02L2 | | | | |
| 3% 1:1 Sytran PC5330 + | 1.5 | 0 | 1.5 | 3 |
| V02L2 | | | | |
| 3% 3:1 Syntran PC | 2.25 | 0.75 | 0 | 3 |
| 5330 + Daitosol 5000 AD | | | | |
| 3% 3:1:1 Syntran PC | 1.5 | 0.5 | 0.5 | 5 |
| 5330 + Daitosol 5000 | | | | |
| AD + V02L2 | | | | |
| 3% V02L2 | 0 | 0 | 3 | 2 |

*amounts of the ingredients in the treatment solutions are expressed in percent total weight of active material in water and ratios are weight ratios

The wash study shows that the materials alone without the inventive composition show poor shampoo resistance and have decreased performance benefits when compared to the association of all three materials.

The wash study shows that each material alone and the combination of the two latex polymers had lower rankings indicating lower shampoo resistance as compared to greater shampoo resistance provided by the composition containing the three materials.

The above examples show that cross linking latex polymers containing carboxylic acid groups (one latex polymer or association of a cationic and a nonionic latex polymer) with polycarbodiimide and the keratin substrate (hair) results in higher stiffness/rigidity, style and shape memory through resiliency against high humidity and shampoo.

8a) Treating hair using one step or two step or reverse step system or regimen.

Hair swatches (IHIP Frizzy Hair) were washed with a cleansing composition.

In the single step (one step) system, the polcarbodiimide and the latex polymer were mixed together and the resulting mixture was applied onto hair. For Step 1 apply 0.1 g/g of hair.

In a two step system, hair swatches were treated first with the polycarbodiimide V02-L2 solution of 2% by weight of active material in water) and then with the latex (solution of 2% by weight of active material in water).

2 Step: first step—apply 0.05 g of solution/g of hair and second step—apply 0.05 g of solution/g of hair. Rough dry for 30 seconds, then blowdry on high heat for 1 minute. Place in humidity chamber.

TABLE 21
1 and 2 STEP Examples (0.1 g of product/g of hair) Latex Blowdry

| | |
|---|---|
| 1 Step- | 2% Syntan 5330 + 2% V02-L2 |
| 2 Step- | 2% V02-L2 first then followed by 2% Syntran 5330 |
| 2 Step Reverse- | 2% Syntran pc 5330 then followed by 2% V02-L2 |

In a reverse step system, hair swatches were treated first with the latex solution, and then treated with the polycarbodiimide solution.

ONE AND TWO STEP Examples (0.2 g of Product/g of Hair) 4% Total Active Weight Latex+Polycarbodiimide—Latex Blowdry

TABLE 22

| | | Lasting frizz in |
|---|---|---|
| | Ease of | Humidity Control (5 hr |
| | Brushing | 80% humidity 25 C.) |
| 1 STEP (combination) | 3 | 2 |
| 2 Step (polycarbodiimide first + | 5 | 5 |
| Syntran PC 5330 second) | | |
| 2 Step Reverse (Syntran PC 5330 | 4 | 4 |
| first + polycarbodiimide | | |
| second) | | |

8b) 2 step styling (airdry)

2 STEP Examples (0.2 g of Product/g of Hair) 4% Latex Airdry

Wash hair (IHIP Frizzy Hair) with Redken Cleansing Cream. Apply solutions (0.1 g/g of hair). For Step 1 apply 0.1 g/g of hair. 2 Step: first step—apply 0.05 g/g of hair and second step—apply 0.05 g/g of hair. Place in humidity chamber.

TABLE 23
1 and 2 STEP Examples (0.1 g of product/g of hair) Latex Airdry

| | |
|---|---|
| 1 Step- | 2% Syntan 5330 + 2% V02-L2 |
| 2 Step- | 2% V02-L2 first then followed by 2% Syntran 5330 |
| 2 Step Reverse- | 2% Syntran pc 5330 then followed by 2% V02-L2 |

TABLE 24

| | Initial frizz | Lasting frizz in |
|---|---|---|
| | in | Humidity Control |
| | Humidty | (5 hr 80% humidity |
| | Control | 25 C.) |
| 1 STEP (0.2 g of latex- | 4 | 2 |
| polycarbodiimide combination) | | |
| 2 Step (0.1 g of polycarbodiimide + | 5 | 5 |
| 0.1 g of Syntran PC 5330) | | |
| 2 Step Reverse (0.1 g of Syntran | 5 | 4 |
| PC 5330 + 0.1 g of | | |
| polycarbodiimide) | | |

The results above show that treating hair according to any one of the three regimens using the above-described compositions resulted in providing styling and/or shaping and/or manageability properties to hair.

In accordance with various embodiments, provided are compositions for treatment of a keratinous substrate comprising: at least one polycarbodiimide compound; and at least one latex polymer selected from acrylate latex polymers; wherein the composition comprises from about 0.2% to about 20.0%, by weight, of the composition of a combined amount of the polycarbodiimide compound and the latex polymer; wherein the combination of polycarbodiimide and latex polymer compounds is present in a ratio of about 1:1 to about 1:5; and wherein the composition includes amounts of each of the polycarbodiimide compound and the latex polymer sufficient to impart one or more of increased hydrophobicity to the keratinous substrate after application thereto and increased hold to the shape or configuration of the keratinous substrate.

In some embodiments, the polycarbodiimide each of the polycarbodiimide compound and the latex polymer is present in a concentration, by weight, of from about 0.1% to about 10%. In some embodiments, each of the polycarbodiimide compound and the latex polymer is present in a concentration, by weight, of from about 0.1% to about 8%.

In some embodiments, the latex polymer is formed from an acrylic acid-based or (meth)acrylic acid-based monomer.

In some embodiments, the latex polymer is selected from Acrylates/Ethylhexyl Acrylate Copolymer, acrylates copolymer, Polyacrylate-2 Crosspolymer, Acrylates/Ethylhexyl Acrylate Copolymer, Acrylates/Hydroxyesters Acrylates Copolymer, Styrene/Acrylates Copolymer, Styrene/Acrylates/Ammonium Methacrylate Copolymer, and mixtures thereof.

In some embodiments, the latex polymer is selected from POLYQUATERNIUM-91 (and) POLYACRYLATE-15, Polyacrylate-15, Polyacrylate 21 (and) Acrylates/Dimethylaminoethyl Methacrylate Copolymer, and acrylate grafted olefin polymer—ACRYLATES/ETHYLHEXYL ACRYLATE/HEMA COPOLYMER (and) ACRYLATES/DIETHYLAMINOETHYL METHACRYLATE/ETHYLHEXYL ACRYLATE COPOLYMER (and) ISODECETH-6 (and) CAPRYLYL GLYCOL (and) SODIUM LAURETH SULFATE.

In some embodiments, the compositions comprise at least two latex polymers.

In some embodiments, the ratio of the polycarbodiimide compound to the latex polymer ranges from between about 1:1 and 1:5.

In some embodiments, the composition produces a film having a Young's modulus ranging from about 175 MPa to about 675 Mpa. In some embodiments, the Young's modulus is greater as the ratio of polycarbodiimide to latex polymer approaches 1:1.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for treatment of a keratinous substrate comprising:
at least one polycarbodiimide compound; and
at least one latex polymer selected from acrylate latex polymers;
wherein the composition comprises from about 0.01% to about 20.0%, by weight, of the composition of a combined amount of the polycarbodiimide compound and the latex polymer;
wherein the composition includes amounts of each of the polycarbodiimide compound and the latex polymer sufficient to impart one or more of increased hydrophobicity to the keratinous substrate after application thereto and increased hold to the shape or configuration of the keratinous substrate.

2. The composition of claim 1, wherein the polycarbodiimide compound has the following formula: 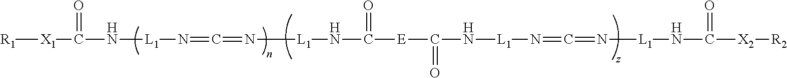
wherein X1 and X2, each independently, represents O, S or NH; R1 and R2, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L1 represents a C1 to C18 divalent aliphatic hydrocarbon group, a C3 to C13 divalent alicyclic hydrocarbon group, a C6 to C14 divalent aromatic hydrocarbon group, a C3 to C12 divalent heterocyclic group, or a C6 to C14 divalent aromatic hydrocarbon group that is not chosen from m-tetramethylxylylene, wherein a plurality of L1 groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

3. The composition according to claim 2, wherein the polycarbodiimide compound is a co-polymer derived from alpha-methylstyryl-isocyanates having the following formula: 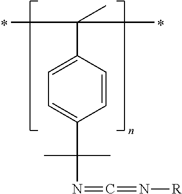
wherein R is an alkyl, cycloalkyl or aryl group having from 1 to 24 carbon atoms.

4. The composition according to claim 2, wherein the polycarbodiimide compound is a compound having the following structure: 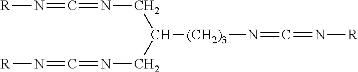
wherein R is an alkyl, cycloalkyl or aryl group.

5. The composition according to claim 4, wherein R is an alkyl, cycloalkyl or aryl group having from 1 to 24 carbon atoms.

6. The composition according to claim 2, wherein each of the polycarbodiimide compound and the latex polymer is present in a concentration, by weight, of from about 0.01% to about 10%.

7. The composition according to claim 2, wherein each of the polycarbodiimide compound and the latex polymer is present in a concentration, by weight, of from about 00.1% to about 8%.

8. The composition according to claim 2, wherein the latex polymer is formed from an acrylic acid-based or (meth)acrylic acid-based monomer.

9. The composition according to claim 8, wherein the latex polymer is selected from Acrylates/Ethylhexyl Acrylate Copolymer, acrylates copolymer, Polyacrylate-2 Crosspolymer, Acrylates/Ethylhexyl Acrylate Copolymer, Acrylates/Hydroxyesters Acrylates Copolymer, Styrene/Acrylates Copolymer, Styrene/Acrylates/Ammonium Methacrylate Copolymer, and mixtures thereof.

10. The composition according to claim 9, wherein the latex polymer is selected from POLYQUATERNIUM-91 (and) POLYACRYLATE-15, Polyacrylate-15, Polyacrylate 21 (and) Acrylates/Dimethylaminoethyl Methacrylate Copolymer, and acrylate grafted olefin polymer—ACRYLATES/ETHYLHEXYL ACRYLATE/HEMA COPOLYMER (and) ACRYLATES/DIETHYLAMINOETHYL METHACRYLATE/ETHYLHEXYL ACRYLATE COPOLYMER (and) ISODECETH-6 (and) CAPRYLYL GLYCOL (and) SODIUM LAURETH SULFATE.

11. The composition according to claim 2, comprising two latex polymers.

12. The composition according to claim 2, wherein the ratio of the polycarbodiimide compound to the latex polymer ranges from between about 1:1 and 1:5.

13. The composition according to claim 12, wherein the composition produces a film having a Young's modulus ranging from about 175 MPa to about 675 Mpa.

14. The composition according to claim 13, wherein the Young's modulus is greater as the ratio of polycarbodiimide to latex polymer approaches 1:1.

15. The composition according to claim 1, comprising a solvent for the polycarbodiimide selected from distilled or de-ionised water, and a solvent for the latex polymer selected from C1-C4 lower alcohols, glycols, polyols, polyol ethers, hydrocarbons, oils, and mixtures thereof.

16. The composition according to claim 1, comprising at least one additive chosen from amino compounds, surfactants (anionic, nonionic, cationic and amphoteric/zwtterionic), and polymers other than the polycarbodiimide and latex polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds is selected from acrylic polymers, non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

17. A method of protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising:
applying to the keratinous substrate a composition according to claim 1 in an amount effective to protect or repair the keratinous substrate,
wherein each of the polycarbodiimide and the latex polymer is provided in a premix comprising at least a solvent, and the premix is applied to the keratinous substrate according to a process selected from a one step process and a multi-step process.

18. A method according to claim 17, wherein the process is selected from:
a one step process, wherein the composition comprising the polycarbodiimide and the latex polymer is provided as a premix, and is prepared by combining the polycarbodiimide and latex polymer and at least a solvent, whereby the premixed composition is applied onto the keratinous substrate;
a one step process, wherein the composition is provided in separate premixes, each separately comprising the polycarbodiimide and the latex polymer, the premixes prepared by combining the polycarbodiimide with at least water to form an aqueous phase, and separately combining the latex polymer with at least an organic solvent to form a non-aqueous phase, whereby at the time of use, the premixed phases are combined and agitated to form an emulsion that is applied onto the keratinous substrate; and
a two-step process, wherein the composition is provided in separate premixes, the premixes prepared by combining the polycarbodiimide with a suitable solvent, and separately combining the latex polymer with a suitable solvent, whereby at the time of use, each of the premixed phases is applied separately to the keratinous substrate in any order, in two sequential applications.

19. A method according to claim 15, wherein the process is a two-step process comprising: the steps selected from (i) applying the latex polymer phase, followed by applying the polycarbodiimide phase, and (ii) applying the polycarbodiimide polymer phase, followed by applying the carboxysilicone phase.

20. An article of manufacture comprising a kit containing, in separately packaged form, the kit comprising:
at least one of:
the composition according to claim 1 wherein the polycarbodiimide and latex polymer are combined in a premix that comprises at least a solvent; and
the composition according to claim 1 wherein the polycarbodiimide and latex polymer are provided in separate packages, comprising a packaged phase premix that comprises polycarbodiimide; and also comprising a packaged phase premix that comprises latex polymer, whereby at the time of use, the premixed phases are combined one of with one another or with a suitable solvent for each of the polycarbodiimide phase and the latex polymer phase; and
optionally, one or more separately packaged solvents,
optionally, one or more separately packaged components selected from surfactants (anionic, nonionic, cationic and amphoteric/zwtterionic), and polymers other than the polycarbodiimide and latex polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds selected from acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.
